(19) United States
(12) Patent Application Publication
(10) Pub. No.: US 2013/0138187 A1
(43) Pub. Date: May 30, 2013

(54) FEEDTHROUGH ASSEMBLY INCLUDING CHIP CAPACITORS

(71) Applicants: Rajesh V. Iyer, Eden Prairie, MN (US); Simon E. Goldman, St. Louis Park, MN (US); Thomas P. Miltich, Otsego, MN (US)

(72) Inventors: Rajesh V. Iyer, Eden Prairie, MN (US); Simon E. Goldman, St. Louis Park, MN (US); Thomas P. Miltich, Otsego, MN (US)

(73) Assignee: MEDTRONIC, INC., Minneapolis, MN (US)

(21) Appl. No.: 13/308,313

(22) Filed: Nov. 30, 2011

(51) Int. Cl. A61N 1/05; H01R 43/00; H01G 4/35

(52) U.S. Cl. 607/116; 361/302; 29/825

(57) ABSTRACT

A feedthrough assembly may include a ferrule defining a ferrule opening, a feedthrough at least partially disposed within the ferrule opening and attached to the ferrule, and a plurality of chip capacitors. In some examples, the feedthrough may include a plurality of feedthrough conductive pathways extending between an externally-facing side of the feedthrough and an internally-facing side of the feedthrough. In some examples, respective ones of the plurality of chip capacitors are electrically connected to respective ones of the plurality of feedthrough conductive pathways and electrically connected to the ferrule.

### TECHNICAL FIELD

The disclosure relates to electrical feedthroughs for implantable medical devices.

### BACKGROUND

Electrical feedthroughs may provide an electrical pathway between an interior of a hermetically-sealed housing of an electronics device to a point outside the housing. For example, implantable medical devices (IMDs), such as implantable stimulation devices, implantable sensing devices, cardiac pacemakers, implantable cardioverter/defibrillators (ICDs) and neuromodulators, may use one or more electrical feedthroughs to make electrical connections between electrical circuitry within the implantable medical device and leads, electrodes, or sensors external to the device within a patient.

### SUMMARY

In general, the disclosure is directed to feedthrough assemblies and techniques for forming feedthrough assemblies. In some examples, the feedthrough assemblies may be used to provide electrical connections between an exterior of a housing of an IMD and an interior of the housing of the IMD. The feedthrough assemblies may be filtered feedthrough assemblies, and may include at least one capacitive filter and/or a capacitive filter array.

In some examples, the disclosure describes a feedthrough assembly that include a plurality of discrete chip capacitors welded to respective ones of conductive pathways in the feedthrough. In some examples, an active lead of a discrete chip capacitor may be welded to a conductive pathway in the feedthrough and a ground lead of the discrete chip capacitor may be welded to a perimeter conductive contact on the feedthrough. In other examples, an active lead of a discrete chip capacitor may be welded to a conductive pathway in the feedthrough and a ground lead of the discrete chip capacitor may be welded to the ferrule.

In a further aspect, the disclosure is directed to a feedthrough assembly that includes a ferrule defining a ferrule opening, a feedthrough at least partially disposed within the ferrule opening and attached to the ferrule, and a plurality of chip capacitors. In accordance with this aspect of the disclosure, the feedthrough may include a plurality of feedthrough conductive pathways extending between an externally-facing side of the feedthrough and an internally-facing side of the feedthrough. In some examples, respective ones of the plurality of chip capacitors are electrically connected to respective ones of the plurality of feedthrough conductive pathways and electrically connected to the ferrule.

In another aspect, the disclosure is directed to an implantable medical device that includes a housing defining an opening and a feedthrough assembly disposed in the opening and attached to the housing. In accordance with this aspect of the disclosure, the feedthrough assembly may include a ferrule opening, a feedthrough at least partially disposed within the ferrule opening and attached to the ferrule, and a plurality of chip capacitors. In accordance with this aspect of the disclosure, the feedthrough may include a plurality of feedthrough conductive pathways extending between an externally-facing side of the feedthrough and an internally-facing side of the feedthrough. In some examples, respective ones of the plurality of chip capacitors are electrically connected to respective ones of the plurality of feedthrough conductive pathways and electrically connected to the ferrule.

In an additional aspect, the disclosure is directed to a method that includes attaching a perimeter wall of a feedthrough to an interior wall of a ferrule to form a hermetic seal between the feedthrough and the ferrule. In accordance with this aspect of the disclosure, the feedthrough may include a plurality of feedthrough conductive pathways extending between an externally-facing side of the feedthrough and an internally-facing side of the feedthrough. The method also may include electrically connecting a respective one of a plurality of chip capacitors between a respective one of the plurality of feedthrough conductive pathways and the ferrule.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a side view that illustrates an example feedthrough assembly.

FIG. 2 is a top view that illustrates an example feedthrough assembly.

FIG. 3 is a cross-sectional view taken along section line A-A of FIG. 2 that illustrates an example configuration of a feedthrough assembly.

FIG. 4 is a flow diagram that illustrates an example technique for forming a feedthrough assembly.

FIG. 5 is a cross-sectional view of another example feedthrough assembly.

FIG. 6 is a flow diagram that illustrates an example technique for forming a feedthrough assembly.

FIGS. 7A and 7B are cross-sectional views taken along section lines A-A and B-B of FIG. 2, respectively, that illustrate an example configuration of a feedthrough assembly.

FIG. 8 is a flow diagram that illustrates an example technique for forming a feedthrough assembly.

FIG. 9 is a side view that illustrates an example feedthrough assembly.

FIG. 10 is a top view that illustrates an example feedthrough assembly.

FIG. 11 is a bottom view that illustrates an example feedthrough assembly.

FIG. 12 is a cross-sectional view taken along section line A-A of FIG. 10 that illustrates an example configuration of a feedthrough assembly.

FIG. 13 is a cross-sectional diagram that illustrates an example chip capacitor.

FIG. 14 is a top view that illustrates an example feedthrough assembly.

FIG. 15 is a bottom view that illustrates an example feedthrough assembly.

FIG. 16 is a cross-sectional view taken along section line A-A of FIG. 14 that illustrates an example configuration of a feedthrough assembly.

FIG. 17 is a top view that illustrates an example feedthrough assembly.

FIG. 18 is a bottom view that illustrates an example feedthrough assembly.

FIG. 19 is a cross-sectional view taken along section line A-A of FIG. 17 that illustrates an example configuration of a feedthrough assembly.

FIG. 20 is a conceptual diagram that illustrates an example feedthrough assembly attached to a housing of an IMD.

### DETAILED DESCRIPTION

In some cases, an IMD is implanted at a different location within the patient than the target tissue that is being stimulated and/or diagnosed. The IMD may be electrically coupled to a lead that includes electrical conductors that extend from the IMD to the electrodes or sensors located at the target tissue. At the IMD, the electrical conductors may be electrically coupled to a conductive pathway through the feedthrough. In some examples, the lead conductors may act as antennae that are affected electromagnetic signals, including electromagnetic interference (EMI). The electromagnetic signals may be transmitted along the lead conductor, through the feedthrough, and to circuitry within the IMD. In some cases, the electromagnetic signals may interfere with normal operation of circuitry within the IMD.

EMI due to stray electromagnetic signals conducted by the lead conductors may be addressed by incorporating a capacitor within the feedthrough assembly. The capacitor may act as a low-pass filter, transmitting relatively high frequency electromagnetic signals to ground (e.g., the housing of the IMD) and passing relatively low frequency signals to circuitry within the IMD. In some examples, the feedthrough assembly may include a multi-conductor feedthrough and a capacitor or capacitor array that accommodates multiple lead conductors. The capacitor or capacitor array may be attached to the multi-conductor feedthrough so that each of the conductive pathways through the multi-conductor feedthrough is electrically coupled to a corresponding conductive path in the capacitor or capacitor array while providing for a hermetic seal around each conductive pathway and between the multi-conductor feedthrough and the ferrule.

In other examples, an IMD may include one or more electrodes formed on a housing of the IMD (e.g., a leadless IMD). In some implementations, a leadless IMD may include a feedthrough assembly through which a conductor that connects the electrodes to circuitry within the leadless IMD passes. The feedthrough assemblies described herein may also be utilized in leadless IMDs.

This disclosure describes various feedthrough assemblies and techniques for forming feedthrough assemblies. The feedthrough assemblies may include a feedthrough and a ferrule. In some examples, the disclosure describes feedthrough assemblies that include a capacitor array, e.g., a plurality of capacitors, electrically connected to the feedthrough. In some examples, the plurality of capacitors may include plurality of discrete capacitors attached to a PB.

In some examples, the disclosure describes a feedthrough assembly that include a plurality of discrete chip capacitors welded to respective ones of conductive pathways in the feedthrough. In some examples, an active lead of a discrete chip capacitor may be welded to a conductive pathway in the feedthrough and a ground lead of the discrete chip capacitor may be welded to a perimeter conductive contact on the feedthrough. In other examples, an active lead of a discrete chip capacitor may be welded to a conductive pathway in the feedthrough and a ground lead of the discrete chip capacitor may be welded to the ferrule.

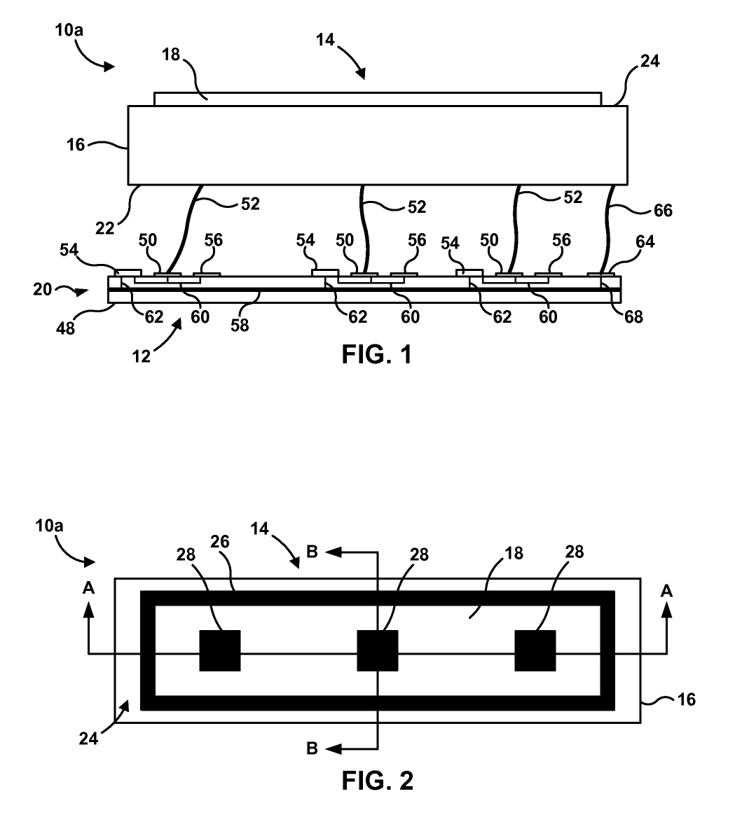

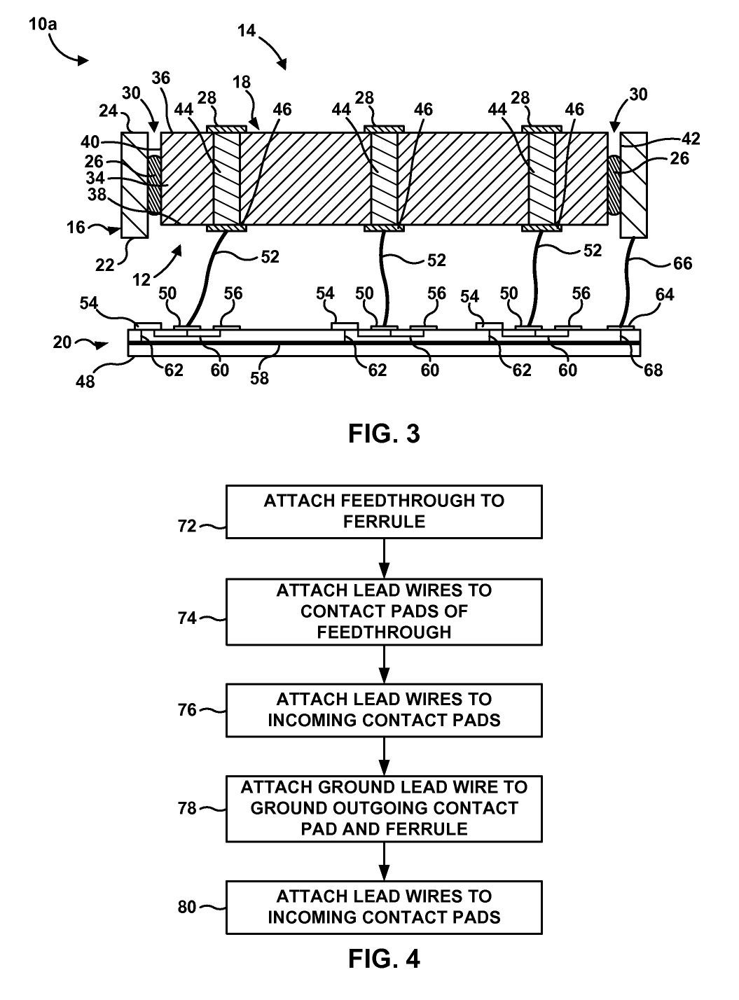

FIG. 1 is a side view of an example feedthrough assembly 10a. Feedthrough assembly 10a includes an internally-facing side 12 and an externally-facing side 14. FIG. 2 shows a top view of feedthrough assembly 10a showing the externally-facing side 14 of feedthrough assembly 10a. FIG. 3 is a cross-sectional view of feedthrough assembly 10a taken along section line A-A of FIG. 2. The terms “internally-facing,” “inwardly,” and the like, when used herein in regards to feedthrough assembly 10, may generally refer to a direction toward the interior of an electronics device (e.g., an IMD) when assembly 10a is incorporated in the electronics device. Conversely, the terms “externally-facing,” “outwardly,” and the like, when used herein in regards to feedthrough assembly 10a generally refer to a direction toward the exterior of the electronics device when assembly 10a is incorporated in the electronics device.

As shown in FIGS. 1-3, feedthrough assembly 10a comprises a ferrule 16, a feedthrough 18, and a capacitor array 20. Feedthrough 12 may be electrically coupled to capacitor array 20 using a plurality of lead wires 52.

Ferrule 16 comprises an internally-facing ferrule side 22 and an externally facing ferrule side 24. Ferrule 16 also defines a ferrule opening 30 that extends between internally-facing side 22 and externally-facing side 24. Feedthrough 12 is at least partially disposed in ferrule opening 30. Ferrule 16 may be configured to be mounted to or within the housing of the electronics device, such as an IMD. In some examples, ferrule 16 may include a flange or other mechanical feature that facilitates mounting of ferrule 16 to or within the housing of the electronics device. Ferrule 16 may be mounted to the IMD housing, for example, by welding or brazing.

In one example, ferrule 16 comprises a material that facilitates mounting of ferrule 16 to the housing of an IMD. For example, the IMD housing may comprise titanium or a titanium alloy, and ferrule 16 may comprise titanium or a titanium alloy that can be welded to the IMD housing. Examples of materials from which ferrule 16 may be formed include niobium; titanium; titanium alloys such as titanium-6Al-4V or titanium-vanadium; platinum; molybdenum; zirconium; tantalum; vanadium; tungsten; iridium; rhodium; rhenium; osmium; ruthenium; palladium; silver; and alloys, mixtures, and combinations thereof. In one example, the material from which ferrule 16 is formed is selected so that ferrule 16 has a coefficient of thermal expansion (CTE) that is compatible with the CTE of feedthrough 18. In this manner, damage resulting from the heating of ferrule 16 and feedthrough 18, such as during the formation of a diffusion bonded, glassed, or brazed joint between ferrule 16 and feedthrough 18, may be reduced or substantially prevented.

Feedthrough 18 may be mounted to ferrule 16 within ferrule opening 30 using a hermetic seal 26 formed between feedthrough 18 and ferrule 16. Hermetic seal 26 may prevent bodily fluids of the patient from passing into the interior of IMD housing between ferrule 16 and feedthrough 18, which could lead to damage to the internal electronics of the IMD. In one example, hermetic seal 26 comprises a braze joint between feedthrough 18 and ferrule 16 (e.g., formed using laser brazing). In other examples, hermetic seal 26 may be formed using diffusion bonding. Examples of materials that may be used to form a hermetic seal 26 include any biocompatible, biostable material capable for forming a hermetic seal 26, such as, gold, a nickel-gold alloy, platinum, and platinum-iridium. Laser sintering of glass may also be used to bond feedthrough 18 to ferrule 16.

As shown in FIG. 3, feedthrough 18 includes a feedthrough substrate 34. Feedthrough substrate 34 defines an externally-facing feedthrough side 36 and an internally-facing feedthrough side 38. Externally-facing feedthrough side 36 is oriented generally opposite to internally-facing feedthrough side 38. Feedthrough substrate 34 also defines a feedthrough perimeter wall 40, which is oriented facing an interior wall 42 of ferrule 16. Feedthrough 18 also includes a plurality of feedthrough conductors 44, which each extend between externally-facing feedthrough side 36 and internally-facing feedthrough side 38. Each of feedthrough conductors 44 is electrically and physically couples to a respective one of externally-facing feedthrough conductive pads 28 and a respective one of internally-facing feedthrough conductive pads 46. Externally-facing feedthrough conductive pads 28 may be disposed on or near externally-facing feedthrough side 36. Internally-facing feedthrough conductive pads 46 may be disposed on or near internally-facing feedthrough side 38. Each of feedthrough conductors 44 may be substantially electrically isolated from the other feedthrough conductors 44. Although FIG. 3 shows an example in which feedthrough 18 includes three externally-facing feedthrough conductive pads 28, three feedthrough conductors 44, and three internally-facing feedthrough conductive pads 46, in other examples, feedthrough 18 may include fewer or more externally-facing feedthrough conductive pads 28, feedthrough conductors 44, and internally-facing feedthrough conductive pads 46 (e.g., one, two, or at least four).

In some examples, feedthrough substrate 34 comprises a ceramic material formed from a single layer. In other examples, feedthrough substrate 34 includes multi-layer ceramic formed from a plurality of generally planar ceramic layers (not shown in FIG. 3). In examples in which feedthrough substrate 34 is formed from multiple ceramic layers, each ceramic layer may be shaped in a green state to have a layer thickness and a plurality of via holes extending there through between an internally facing layer surface and an externally facing layer surface. The ceramic layers then may be coupled together, such as by laminating the layers together, and may be cofired together so that the layers form a substantially monolithic feedthrough substrate 34. In some examples, the via holes of each ceramic layer may be substantially aligned to form generally cylindrical passages that are filled with an electrically conductive material to form conductors 44.

In some examples, feedthrough substrate 34 may include a high-temperature cofired ceramic (HTCC) material, e.g., a ceramic that is sintered at a temperature of at least about 1300° C., for example a material that is sintered at a temperature of at least about 1600° C. In some embodiments, HTCC uses 1) an electrical insulator that includes alumina and may include oxides of Si (silica), Ca (calcia), Mg (magnesia), Zr (zirconia), and the like and 2) an electrical conductor, such as platinum or Pt—Ir (which may form feedthrough conductors 44). The assembly of the electrical insulator and electrical conductor can be fired (sintered) above 1000° C., such as about 1600° C. In this sintering process, polymeric binders may be driven off and the particles forming the ceramic and metal coalesce and fuse. Grains may diffuse together forming larger grains at the expense of smaller grains.

In one example, feedthrough substrate 34 comprises an HTCC liquid-phase, sintered alumina with platinum metallization. In one example, feedthrough substrate 34 may comprise at least about 70% alumina, for example at least about 90% alumina having a sintering temperature of between about 1550° C. and about 1600° C. In some examples, feedthrough substrate 34 consists essentially of a HTCC, and in some examples, feedthrough substrate 34 consists of a HTCC.

Examples of materials and methods for making a co-fired ceramic substrate are described in the commonly assigned U.S. Provisional Patent Application Ser. No. 61/530,249, filed on Sep. 1, 2011; the commonly assigned U.S. Provisional Patent Application Ser. No. 61/238,515, filed on Aug. 31, 2009; the commonly assigned United States Patent Application Publication No. 2011/0048770, filed on Jan. 26, 2010, the commonly assigned U.S. Pat. No. 6,414,835, issued on Jul. 2, 2002, the commonly-assigned U.S. Pat. No. 6,660,116, issued on Dec. 9, 2003, U.S. patent application Ser. No. 13/196,661, filed on Aug. 2, 2011, U.S. patent application Ser. No. 13/196,683, filed on Aug. 2, 2011, and U.S. patent application Ser. No. 13/196,695, filed on Aug. 2, 2011, the entire disclosures of which are incorporated herein by reference.

Conduction of electrical signals between externally-facing feedthrough side 36 and internally-facing feedthrough side 38 may be accomplished using externally-facing feedthrough conductive pads 28, feedthrough conductors 44 and internally-facing feedthrough conductive pads 46. Together, a respective one of externally-facing feedthrough conductive pads 28, a respective one of feedthrough conductors 44, and a respective one of internally-facing feedthrough conductive pads 46 form a feedthrough conductive pathway between externally-facing feedthrough side 36 and internally-facing feedthrough side 38. The electrically conductive pathways provide for an electrical pathway for electrical signals to be transmitted across feedthrough substrate 34, such as stimulation signals transmitted from electronics within an IMD housing for stimulation of a target tissue, or bioelectric signals sensed proximate a target tissue that are transmitted into the IMD housing for analysis by IMD electronics.

Feedthrough conductors 44 may comprise a conductive material, such as a metal or alloy, that substantially fills a passageway that extends through feedthrough substrate 34. In one example, a hermetic seal is formed at the interface between each of feedthrough conductors 44 and feedthrough substrate 34. The hermetic seal may be formed by many methods, such as by forming a braze joint between the material that forms conductor 44 and the material that forms feedthrough substrate 34. In one example, described in more detail below, the hermetic seal is formed by cofiring the materials that form feedthrough substrate 34 and feedthrough conductors 44 so that the material that forms conductors 44 bonds with the material that forms feedthrough substrate 34.

Each electrically conductive pathway also may include an internally-facing feedthrough conductive pad 46 at internally-facing side 38. Each internally-facing feedthrough conductive pad 46 may provide a contact area to provide for electrical and/or mechanical coupling between the respective electrically conductive pathway and a respective one of lead wires 52. In some examples, each internally-facing feedthrough conductive pad 46 is electrically and mechanically coupled to a corresponding one of feedthrough conductors 44.

Each electrically conductive pathway may also include an externally-facing feedthrough conductive pad 28 at externally-facing side 36. Each conductive pad 28 may provide contact area to provide for electrical and/or mechanical coupling of a conductor, such as a lead conductor for an IMD, to the respective electrically conductive pathway (e.g., the conductive pad 28). In some examples, each externally-facing feedthrough conductive pad 28 is electrically and mechanically coupled to a corresponding one of conductors 44.

In some examples, conductors 44 and conductive pads 28, 46 each include an electrically conductive material, such as an electrically conductive metal or alloy. Examples of electrically conductive materials that may be used for conductors 44 and/or conductive pads 28, 46 include, but are not limited to, transition metals (e.g., noble metals), rare earth metals (e.g., actinide metals and lanthanide metals), alkali metals, alkaline-earth metals, and rare metals. Examples of materials that may be used to form conductors 44 and/or conductive pads 28, 46 include, but are not limited to, copper (Cu), silver (Ag), gold (Au), platinum (Pt), palladium (Pd), niobium (Nb), iridium (Ir), titanium (Ti), tungsten (W), molybdenum (Mb), zirconium (Zr), osmium (Os), tantalum (Ta), vanadium (V), rhodium (Rh), rhenium (Re), and ruthenium (Ru), platinum-gold alloys, platinum-iridium alloys, platinum-palladium alloys, gold-palladium alloys, titanium alloys, such as Ti-6Al-4V, Ti-45Nb, Ti-15Mo or titanium-vanadium, tungsten-molybdenum alloys, and alloys, mixtures, and combinations thereof.

With respect to internally-facing feedthrough conductive pads 46, in some examples, the material and structure of conductive pads 46 may be selected to support bonding of a corresponding electrical connection (such as one of lead conductors 52) to provide electrical and mechanical coupling between respective ones of internally-facing feedthrough conductive pad 46 and respective ones of incoming contact pads 50 on capacitor array 20.

With respect to externally-facing feedthrough conductive pads 28, the material and structure of conductive pads 28 may be selected to support welding of a conductor, such as a wire or conductor used in a lead for an IMD, to external surfaces of respective ones of conductive pads 28. Examples of materials that may be used in an IMD lead conductor that may be welded to conductive pads 28 include, but are not limited to, niobium (Nb), a MP35N or MP35NLT nickel-based alloy, silver core Co—Cr—Ni alloy, tantalum, silver core Ta, Ti, Ti-45Nb, Ti—Mo alloys, and alloys meeting ASTM standard F562. Examples of processes that may be used to attach the lead conductor to conductive pads 28 include, but are not limited to, laser welding, parallel gap welding, thermosonic bonding, diffusion bonding, ultrasonic welding, opposed gap welding, laser brazing, step gap resistance welding, brazed interposer, percussion arc welding, or soldering (conventional or laser).

In some examples in which feedthrough substrate 34 comprises a HTCC material, feedthrough conductors 44 and/or externally-facing feedthrough conductive pads 28 and/or internally-facing feedthrough conductive pads 46 may include a conductive paste that is used to fill passageways extending from externally-facing feedthrough side 36 and internally-facing feedthrough side 38 to form conductors 44. The conductive paste may comprise, for example, a metallic paste that is applied to the passageways, for example a platinum-containing paste, a tungsten-containing paste, Nb-containing paste, Ta-containing paste, Au-containing paste, or a molymanganese-containing paste. Such materials may be biocompatible and biostable materials. In one example, the metallic paste primarily comprises a metallic powder, such as platinum powder, and an additive to promote bonding with the material of feedthrough substrate 34. The additive may additionally or alternatively provide for thermal expansion compatibility between the conductive paste used to form feedthrough conductors 44 (and/or pads 28, 46) and the HTCC material of feedthrough substrate 34. In one example, the additive comprises alumina, so that the metallic paste may comprise, for example, a majority of metallic powder, such as platinum powder, and a minority of alumina powder or particles mixed therein.

In some examples, feedthrough conductors 44 and/or pads 28, 46 formed from a conductive paste, such as a platinum and alumina containing paste, and a feedthrough substrate 34 comprising an HTCC material, such as a sintered alumina, are cofired together, e.g., at a temperature of around 1600° C., so that the conductive paste and HTCC material bond together and form hermetic seal.

Conductive pathways through feedthrough 18 (e.g., including one of externally-facing feedthrough conductive pads 28, one of feedthrough conductors 44, and one of internally-facing feedthrough conductive pads 46) are electrically connected to capacitor array 20. Capacitor array 20 includes a PB 48, a plurality of incoming contact pads 50 attached to PB 48, a plurality of capacitors 54 attached to PB 48, and a plurality of outgoing contact pads 56 attached to PB 48. Incoming contact pads 50 and outgoing contact pads 56 may be formed on an electrically conductive material and may be configured to allow attachment of lead wires 52 and wires that extend to circuitry within the IMD, respectively. For example, incoming contact pads 50 and/or outgoing contact pads 56 may be formed of copper, silver, aluminum, gold, platinum, or the like.

Capacitors 54 may include any type of capacitor that may be surface mounted or through-hole mounted to PB 48. In some examples, the type of capacitor 54 may be classified by the dielectric material used in capacitor 54. Capacitor 54 may include a paper, plastic, glass, mica, or ceramic solid dielectric material, or air as a dielectric material. Capacitors 54 may be mounted to PB 48 using a surface mounting technique, such as soldering leads of capacitors 54 to conductive pads on a surface of PB 48. Alternatively, capacitors 54 may be mounted to PB 48 using through-hole mounting.

In some examples, capacitors 54 provide for filtering of electrical signals that are conducted through the corresponding feedthrough conductors 44 to which the capacitors 54 are electrically connected. For example, each of capacitors 54 may provide for filtering of current induced in an IMD lead by external electromagnetic fields so that the induced current is not inadvertently interpreted by the IMD circuitry as a signal, such as a telemetry signal. In some implementations, capacitors 54 may be selected to function as low-pass filters, which pass electrical signals with a frequency below about 5 megahertz (MHz) and filter signals with a frequency above about 20 MHz. In this way, capacitors 54 may reduce or substantially eliminate electrical interference due to signals inadvertently sensed and conducted by the lead conductors, and may facilitate or protect operation of the electronics in the IMD.

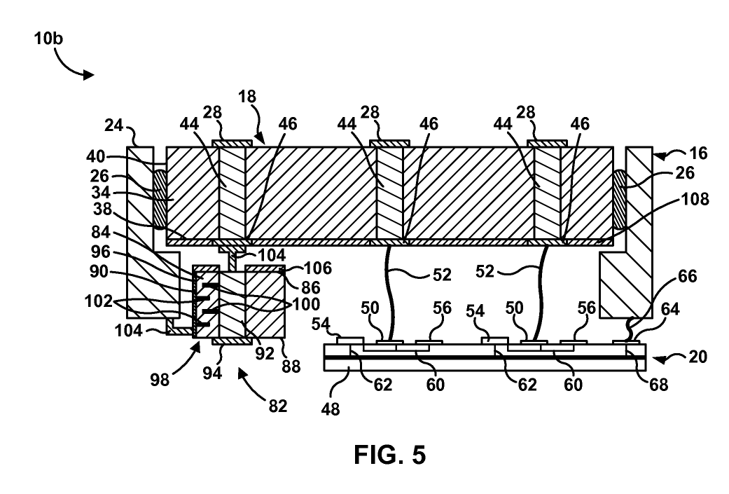

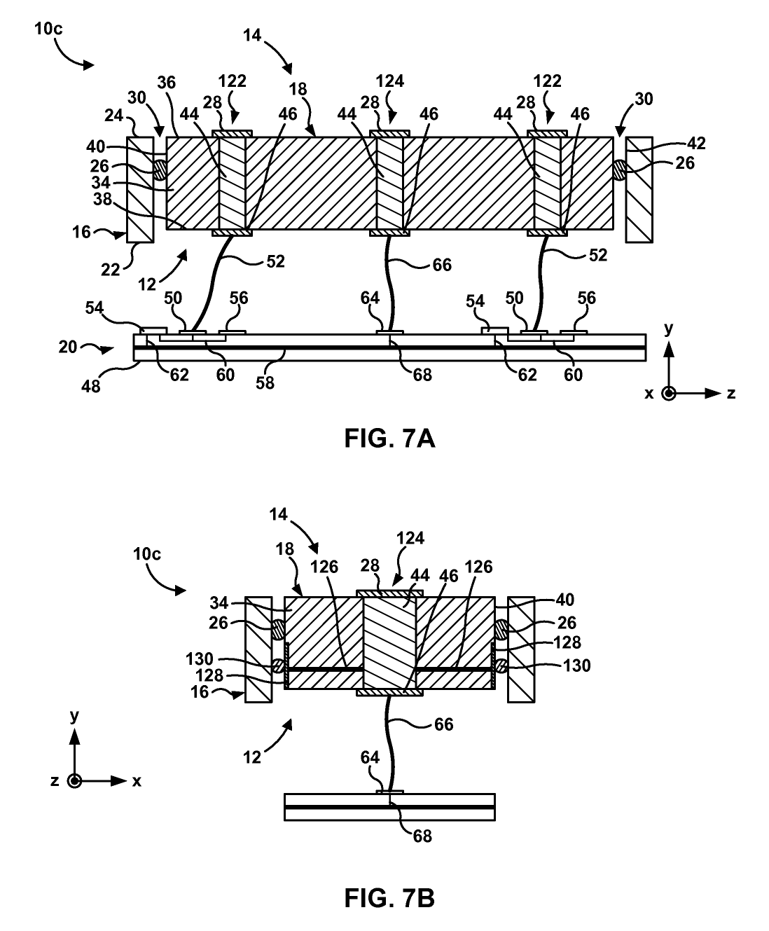

Although FIG. 3 illustrates three capacitors 54, three incoming contact pads 50, and three outgoing contact pads 56, in other examples, feedthrough assembly 10a may include more than three capacitors 54, incoming contact pads 50, and/or outgoing contact pads 56 or fewer than three capacitors 54, incoming contact pads 50, and/or outgoing contact pads 56. In some examples, feedthrough assembly 10a include the same number of each of capacitors 54, incoming contact pads 50, and/or outgoing contact pads 56 as the number of conductive pathways through feedthrough 18. In other examples, feedthrough assembly 10 may include a different number of capacitors 54, incoming contact pads 50, and/or outgoing contact pads 56 than the number of conductive pathways through feedthrough 18 (see FIGS. 5 and 7A).

PB 48 may include a substrate that includes an electrically insulating material, such as an epoxy, a polytetrafluoroethylene, or a polyester. PB 48 also may include a plurality of electrical traces formed of an electrically conductive material, such as gold, silver, aluminum, or copper. The electrical traces may be formed in planes within PB 48 (e.g., in a plane between layers of electrically insulating material (in the x-y plane of FIG. 3) or on a surface of PB 48. In some examples, at least some of the electrical traces may extend in the z-axis direction shown in FIG. 3. The electrical traces may be formed by any of a variety of methods, such as silk screen printing, laser resist ablation, or the like.

The electrical traces may include active electrical traces 60 and ground electrical traces 62. Active electrical traces 60 electrically connect respective ones of incoming contact pads 50 to an active electrode of respective ones of capacitors 54. Active electrical traces 60 also may electrically connect respective ones of incoming contact pads 50 to respective ones of outgoing contact pads 56 (either in series with capacitors 54 or in parallel with capacitors 54). In this way, active electrical traces 60 may provide an electrical path for electrical signals between feedthrough 18 and circuitry within the IMD.

Ground electrical traces 62 may electrically connect ground electrodes of respective ones of capacitors 54 to ground plane 58. Ground plane 58 may be an electrical trace or an electrically conductive layer of material disposed in a plane within or on a surface of PB 48. For example, ground plane 58 may be formed of copper, gold, silver, aluminum, or platinum.

Ground plane 58 may be electrically connected to an electrical ground. In some examples, as shown in FIGS. 1 and 3, ground plane 58 is electrically connected to an outgoing ground contact pad 64 by a ground contact pad trace 68. Outgoing ground contact pad 64 may be formed on a surface of PB 48, and may include an electrically conductive material, such as copper, gold, silver, aluminum, platinum, or the like.

Ground lead wire 66 may be electrically connected to outgoing ground contact pad 64. For example, ground lead wire 66 may be soldered or welded to outgoing ground contact pad 64. Ground lead wire 66 may be electrically connected to ferrule 16 using, for example, soldering or welding. In some examples, as described below with respect to FIG. 20, ferrule 16 may be electrically connected to a housing of an IMD, and the housing may function as an electrical ground. In this way, conductor array 20 may provide filtering of electrical signals conducted through feedthrough 18.

FIG. 4 is a flow diagram that illustrates an example technique for forming feedthrough assembly 10a. The technique illustrated in FIG. 4 may include attaching feedthrough 18 to ferrule 16 (72). Feedthrough 18 may be attached to ferrule 16 using any technique that forms hermetic seal 26 between feedthrough 18 and ferrule 16. For example, ferrule 16 and feedthrough 18 may be connected using brazing, diffusion bonding, laser sintering of glass, or the like. Hermetic seal 26 may be formed using a biocompatible, biostable material. Examples of materials that may be used to form a hermetic seal 26 include gold, a nickel-gold alloy, platinum, platinum-iridium, or a biocompatible glass.

The technique of FIG. 4 also may include attaching lead wires 52 to feedthrough 18 (74). In some examples, lead wires 52 may be attached to internally-facing feedthrough conductive pads 46, as shown in FIG. 3. In other examples, feedthrough 18 may not include internally-facing feedthrough conductive pads 46, and lead wires 52 may be attached to feedthrough conductors 44. As described above, lead wires 52 may be attached to internally-facing feedthrough conductive pads 46 (or feedthrough conductors 44) by soldering, welding, or the like.

The technique of FIG. 4 also may include attaching lead wires 52 to incoming contact pads 50 of capacitor array 20 (76). As described above, lead wires 52 may be attached to internally-facing feedthrough conductive pads 46 (or feedthrough conductors 44) by soldering, welding, or the like. In other examples, the method may not include attaching lead wires 52 to feedthrough 18 (74) and attaching lead wires 52 to incoming contact pads 50 of capacitor array 20 (76). Instead, the method may include directly electrically connecting internally-facing feedthrough conductive pads 46 (or feedthrough conductors 44) to incoming contact pads 50 using, for example, soldering, welding, brazing, or the like. In some examples, internally-facing feedthrough conductive pads 46 (or feedthrough conductors 44) and/or incoming contact pads 50 may be configured to allow direct connection between internally-facing feedthrough conductive pads 46 (or feedthrough conductors 44) and incoming contact pads 50. For example, internally-facing feedthrough conductive pads 46 may extend sufficient far past internally-facing feedthrough side 38 to allow direct contact with incoming contact pads 50.

The technique of FIG. 4 also may include attaching ground lead wire 66 to ferrule 16 and outgoing ground contact pad 64 (78). As described above, ground lead wire 66 may be attached to ferrule 16 and/or outgoing ground contact pad 64 by soldering, welding, or the like. In other examples, the method may not include attaching ground lead wire 66 to ferrule 16 and outgoing ground contact pad 64 (78). Instead, the method may include directly electrically connecting ferrule 16 to outgoing ground contact pad 64 using, for example, soldering, welding, brazing, or the like. In some examples, ferrule and/or outgoing ground contact pad 64 may be configured to allow direct connection between ferrule 16 and outgoing ground contact pad 64. For example, ferrule 16 may extend sufficient far past internally-facing feedthrough side 38 of feedthrough 18 to allow direct contact with outgoing ground contact pad 64.

In some examples, the technique of FIG. 4 optionally may include mechanically attaching capacitor array 20 to ferrule 16 and/or feedthrough 18 by mechanical means other than lead wires 52 and ground lead wire 66 (80). For example, capacitor array 20 may be mechanically attached to ferrule 16 and/or feedthrough 18 using an adhesive. The adhesive may maintain relative positioning between capacitor array 20 and ferrule 16 and/or feedthrough 18, and may provide increased mechanical integrity to feedthrough assembly 10a. In other examples, capacitor array 20 may be connected to feedthrough 18 and ferrule 16 only by lead wires 52 and ground lead wire 66, respectively.

The example feedthrough assembly 10a illustrated in FIGS. 1-3 includes capacitor array 20 with capacitors 54 for each conductive pathway through feedthrough 18. However, in other examples, capacitor array 20 may include capacitors for only some of the conductive pathways through feedthrough 18 and other conductive pathways through feedthrough 18 may be electrically connected to other capacitive filters. FIG. 5 illustrates a cross-sectional view of an example feedthrough assembly 10b that includes a co-fire capacitive filter 82 electrically coupled to a first conductive pathway through feedthrough 18 and a capacitor array 20 electrically coupled to second and third conductive pathways through feedthrough 18.

Feedthrough 18 and capacitor array 20 may be similar or substantially the same as feedthrough 18 and capacitor array 20 described with reference to FIGS. 1-3. However, instead of including the same number of incoming contact pads 50, capacitors 54, and outgoing contact pads 56 as feedthrough 18 has conductive pathways, capacitor array 20 includes two incoming contact pads 50, capacitors 54, and outgoing contact pads 56 and feedthrough 18 includes three conductive pathways.

In some examples, feedthrough 18 may include a feedthrough electrically insulating layer 108 on internally-facing feedthrough side 38. Feedthrough electrically insulating layer 108 may reduce or prevent high-voltage arcing between feedthrough 18 and co-fire capacitive filter 82 and/or between feedthrough 18 and capacitor array 20. Feedthrough electrically insulating layer 108 may include an electrically insulating material, such as an electrically insulating polymer formed on internally-facing feedthrough side 38. In one example, feedthrough electrically insulating layer 108 comprises a polyimide polymer with a glass transition temperature of greater than about 400° C. In some examples, feedthrough electrically insulating layer 108 may comprise a low temperature cofired ceramic material or a HTCC material.

Feedthrough assembly 10b includes co-fire capacitive filter 82, which is electrically coupled to a first conductive pathway through feedthrough 18. The conductive pathway includes one of externally-facing feedthrough conductive pads 28, one of feedthrough conductors 44, and one of internally-facing feedthrough conductive pads 46. As shown in FIG. 5, co-fire capacitive filter 82 is electrically coupled to feedthrough 18 and ferrule 16 using a lead frame assembly. The lead frame assembly may include electrically conductive leads 104. A first one of electrically conductive leads 104 electrically connects one of internally-facing feedthrough conductive pads and capacitive filter conductor 92. A second one of electrically conductive leads 104 electrically connects a capacitive filter perimeter conductive contact 96 and ferrule 16.

Each of electrically conductive leads 104 may be formed of an electrical conductive metal, such as niobium; titanium; titanium alloys such as titanium-6Al-4V or titanium-vanadium; platinum; molybdenum; zirconium; tantalum; vanadium; tungsten; iridium; rhodium; rhenium; osmium; ruthenium; palladium; silver; and alloys, mixtures, and combinations thereof. In various examples, electrically conductive leads 104 may be soldered, welded, brazed, or fired to internally-facing feedthrough conductive pads 46, capacitive filter conductor 92, capacitive filter perimeter conductive contact 96 and/or ferrule 16. In some examples, electrically conductive leads 104 may include bare metal (e.g., with no electrical insulation formed on a surface of electrically conductive leads 104). In other examples, at least one of electrically conductive leads 104 may include electrical insulation formed on a surface of the at least one of electrically conductive leads 104, such as an electrically insulating polymer.

Although FIG. 5 illustrates co-fire capacitive filter 82 being attached to ferrule 16 and feedthrough 18 using electrically conductive leads 104, in other examples, co-fire capacitive filter 82 may be attached and electrically connected to ferrule 16 and/or feedthrough 18 using other mechanisms, such as a thick film conductive paste. In some examples, the thick film conductive paste may include a silver-palladium (Ag—Pd) mixture or alloy. In some implementations, the Ag—Pd mixture or alloy may include about 70 weight percent (wt. %) Ag and about 30 wt. % Pd. In some examples, the Ag—Pd mixture or alloy may also include glass frit (e.g., glass particles mixed in the Ag—Pd mixture or alloy). In some examples, the glass frit includes a zinc borosilicate glass particles, and may be dispersed in an organic binder.

Thick film conductive paste 48 may be applied to any desired thickness. In some examples, the thickness of at least one of thick film conductive paste 48a, 48b, 48c, 48d, 48e is about 0.00254 millimeters (mm; (about 0.0001 inch).

In some examples, the thick film conductive paste may form the only mechanical connections between feedthrough 18 and capacitive filter array 20 and/or between capacitive filter array 20 and ferrule 16. The thick film conductive paste may possess sufficient mechanical strength to function as the only mechanical connection between feedthrough 18 and capacitive filter array 20 and between capacitive filter array 20 and ferrule 16 (e.g., after firing to convert the thick film conductive paste from a paste to a relatively solid material). In some examples, the thick film conductive paste may be disposed between internally-facing feedthrough conductive pads 46 and capacitive filter conductor 92. In some examples, the thick film conductive paste may be disposed between capacitive filter perimeter conductive contact 96 and an interior wall of ferrule 16.

Co-fire capacitive filter 82 includes a filter substrate 84, which defines an externally-facing filter side 86, an internally-facing filter side 88, and a capacitive filter perimeter wall 90. Co-fire capacitive filter 82 also includes a capacitive filter conductive pathway, which includes capacitive filter conductor 92 and internally-facing filter conductive pad 94. In some examples, the capacitive filter conductive pathway also may include an externally-facing filter conductive pad (not shown in FIG. 5). Although FIG. 5 illustrates an example co-fire capacitive filter 82 that includes one capacitive filter conductive pathway, in other examples, co-fire capacitive filter 82 may include more than one capacitive filter conductive pathway (e.g., at least two).

Co-fire capacitive filter 82 further includes a capacitive filter 98 defined within filter substrate 84, which is electrically connected to the capacitive filter conductive pathway. The capacitive filter conductive pathways provides an electrical pathway for electrical signals to be transmitted through co-fire capacitive filter 82, such as stimulation signals transmitted from electronics within an IMD housing for stimulation of a target tissue or bioelectric signals sensed proximate a target tissue that are transmitted into the IMD housing for analysis by IMD electronics. Co-fire capacitive filter 82 filters the electrical signals transmitted through co-fire capacitive filter 82 using capacitive filter 64.

Filter substrate 84 may be formed of a ceramic material. In some examples, filter substrate 84 may be formed from a single layer. In other examples, filter substrate 84 includes a multi-layer ceramic formed from a plurality of generally planar ceramic layers (not shown in FIG. 5). In examples in which filter substrate 84 is formed from multiple ceramic layers, each ceramic layer may be shaped in a green state to have a layer thickness and a plurality of via holes extending there through between an internally facing layer surface and an externally facing layer surface. The ceramic layers then may be coupled together, such as by laminating the layers together, and may be cofired together so that the layers form a substantially monolithic filter substrate 84. In some examples, the passageways of each ceramic layer may be substantially aligned to form generally cylindrical passages that are filled with an electrically conductive material to form capacitive filter conductor 92.

In some examples, filter substrate 84 may comprise a high-temperature cofired ceramic (HTCC) material, e.g., a ceramic that is sintered at a temperature of at least about 1300° C., for example a material that is sintered at a temperature of at least about 1600° C. In some embodiments, HTCC uses 1) an electrical insulator that includes barium titanate (BaTiO₃) or alumina (Al₂O₃) and may include oxides of Si (silica), Ca (calcia), Mg (magnesia), Zr (zirconia), and the like and 2) an electrical conductor, such as platinum or Pt—Ir. The assembly of the electrical insulator and electrical conductor can be fired (sintered) above 1000° C., such as about 160° C. In this sintering process, polymeric binders may be driven off and the particles forming the ceramic and metal coalesce and fuse. Grains may diffuse together forming larger grains at the expense of smaller grains.

Co-fire capacitive filter 82 also capacitive filter conductive pathway. As described above, the capacitive filter conductive pathway includes capacitive filter conductor 92 and internally-facing filter conductive pad 94. Capacitive filter conductor 92 and internally-facing filter conductive pad 94 each may include an electrically conductive material, such as an electrically conductive metal or alloy. Examples of electrically conductive materials that may be used for conductor 92 and/or conductive pad 94 include, but are not limited to, transition metals (e.g., noble metals), rare earth metals (e.g., actinide metals and lanthanide metals), alkali metals, alkaline-earth metals, and rare metals. Examples of materials that may be used to form conductor 92 and/or conductive pad 94 include, but are not limited to, copper (Cu), silver (Ag), gold (Au), platinum (Pt), palladium (Pd), niobium (Nb), iridium (Ir), titanium (Ti), tungsten (W), molybdenum (Mb), zirconium (Zr), osmium (Os), tantalum (Ta), vanadium (V), rhodium (Rh), rhenium (Re), and ruthenium (Ru), platinum-gold alloys, platinum-iridium alloys, platinum-palladium alloys, gold-palladium alloys, titanium alloys, such as Ti-6Al-4V, Ti-45Nb, Ti-15Mo or titanium-vanadium, tungsten-molybdenum alloys, and alloys, mixtures, and combinations thereof.

With respect to internally-facing filter conductive pads 94, in some examples, the material and structure of conductive pads 94 may be selected to support an electrical connection to a corresponding electrical conductor that extends between internally-facing filter conductive pads 94 and circuitry of the IMD (e.g., sensing circuitry, therapy delivery circuitry, or the like).

In some examples, a filter electrically insulating layer 106 may be placed between feedthrough 18 and co-fire capacitive filter 82 in order to reduce or prevent high-voltage arcing between feedthrough 18 and filter 82. Filter electrically insulating layer 106 may also be provided to prevent arcing between the conductive path (which may be continuous between the externally-facing feedthrough conductive pads 28 and filter array 20) and ferrule 16, or between the conductive path and filter perimeter conductive contact 96, as any direct line of sight between the conductive two electrically conductive materials may cause surface arcing. In this sense, filter electrically insulating layer 106 may reduce or substantially prevent surface arcing.

Filter electrically insulating layer 106 may include an electrically insulating material, such as an electrically insulating polymer formed on externally-facing filter side 86. In one example, filter electrically insulating layer 106 comprises a polyimide polymer with a glass transition temperature of greater than about 400° C. In some examples, filter electrically insulating layer 106 may comprise a low temperature cofired ceramic material or a HTCC material. Although not shown in FIG. 5, in some examples, filter electrically insulating layer 106 may additionally or alternatively be formed on internally-facing filter array side 88 and/or externally-facing feedthrough side 36.

At least a portion capacitive filter conductor 92 is electrically connected to capacitive filter 98, which provides for filtering of electrical signals that are conducted through the conductor 92. For example, capacitive filter 98 may provide for filtering of current induced in an IMD lead by external electromagnetic fields so that the induced current is not inadvertently interpreted by the IMD circuitry as a signal, such as a telemetry signal. In some examples, capacitive filter 64 comprises a plurality of layers (not shown) of ceramic, such as barium titanate, with conductive active electrodes 100 and ground electrodes 102 formed on the layers, such as by printing the material of electrodes 100, 102, for example silver, silver-palladium, or silver-platinum, onto the layers before stacking and laminating the layers. Active electrodes 100 are electrically coupled to capacitive filter conductor 92. Ground electrodes 102 are electrically connected to capacitive filter perimeter conductive contact 96.

Capacitive filter perimeter conductive contact 96 may extend along at least a portion of the length of capacitive filter perimeter 90, as shown in FIG. 5, so that each ground electrode 68 is electrically coupled to capacitive filter perimeter conductive contact 96. Capacitive filter perimeter conductive contact 96 is electrically coupled to a common ground so that the EMI signals being filtered by co-fire capacitive filter 82 are grounded. In some examples, shown in FIG. 5, capacitive filter perimeter conductive contact 96 is grounded by being electrically coupled to ferrule 16, which in turn is electrically coupled to the IMD housing. Capacitive filter perimeter conductive contact 96 may be formed of an electrically conductive metal or alloy, such as, for example, silver, silver-palladium, or silver-platinum.

The example of co-fire capacitive filter 82 in FIG. 5 includes a single conductive pathway (that includes capacitive filter conductor 92 and internally-facing filter conductive pad 94) and a single capacitive filter 98. However, in other examples, co-fire capacitive filter 82 may include a plurality of conductive pathways and a corresponding number of capacitive filters 98. Respective ones of the plurality of conductive pathways may be connected to respective ones of the conductive pathways in feedthrough 18. In this way, a co-fire capacitive filter 82 may be used to filter signals from a plurality of conductive pathways in feedthrough 18.

In some examples, co-fire capacitive filter 82 may provide capacitive filtering for the conductive pathways to which co-fire capacitive filter 82 is electrically attached with a higher signal-to-noise ratio than capacitors 54. By using both co-fire capacitive filter 82 and capacitor array 20, feedthrough assembly 10b may enable flexibility in design and manufacture. For example, in some implementations, co-fire capacitive filter 82 may have better filter characteristics in the stop band (i.e., the frequencies that co-fire capacitive filter 82 does not pass), such as higher insertion loss. However, co-fire capacitive filter 82 may be more expensive than capacitor array 20. Conversely, in some examples, capacitor array 20 may be less expensive than co-fire capacitive filter 82 but may provide less desirable high-frequency filtering characteristics. For example, capacitors 54 in capacitor array 20 may have a lower insertion loss than co-fire capacitive filter 82.

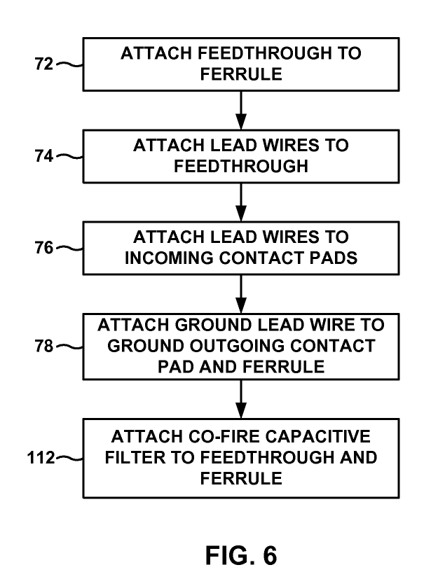

FIG. 6 is an example technique for forming feedthrough assembly 10b shown in FIG. 5. The technique illustrated in FIG. 6 includes attaching feedthrough 18 to ferrule 16 (72), attaching lead wires 52 to feedthrough 18 (74), attaching lead wires 52 to incoming contact pads 50 of capacitor array 20 (76), and attaching ground lead wire 66 to ferrule 16 and outgoing ground contact pad 64 (78). Each of these steps may be similar or the same as the corresponding steps described with respect to FIG. 4. Additionally, the technique of FIG. 6 includes attaching co-fire capacitive filter 82 to feedthrough 18 and ferrule 16 (112).

In some examples, co-fire capacitive filter 82 may be attached to feedthrough 18 and ferrule 16 using electrically conductive leads 104. In some of these examples, attaching co-fire capacitive filter 82 to feedthrough 18 and ferrule 16 (112) may include attaching electrically conductive leads 104 to co-fire capacitive filter 82. In some examples, attaching electrically conductive leads 104 includes one of electrically conductive leads 104 directly to capacitive filter conductor 92 and capacitive filter perimeter conductive contact 96. In other examples, co-fire capacitive filter 82 may include an externally-facing filter conductive pad, and the electrically conductive lead 104 may be attached to the externally-facing filter conductive pad.

Electrically conductive leads 104 may be attached to co-fire capacitive filter 82 using a variety of techniques. For example, electrically conductive leads 104 may be attached to co-fire capacitive filter 82 using laser welding, parallel gap welding, thermosonic bonding, diffusion bonding, ultrasonic welding, opposed gap welding, laser brazing, step gap resistance welding, percussion arc welding, or soldering (conventional or laser).

In other examples, electrically conductive leads 104 may be attached to co-fire capacitive filter 82 (e.g., capacitive filter conductor 92 and/or capacitive filter perimeter conductive contact 96) using a firing process. In a firing process, co-fire capacitive filter 82 and electrically conductive leads 104 may be heated to a temperature between about 700° C. and about 850° C. for between about 30 minutes and about 60 minutes, with about 10 minutes of substantially constant temperature at the peak temperature. The heating process may result in a mechanical connection between electrically conductive leads 104 and capacitive filter conductor 92 and capacitive filter perimeter conductive contact 96.

Once electrically conductive leads 104 have been attached to co-fire capacitive filter 82, co-fire capacitive filter array 82 (including electrically conductive leads 104) may be positioned in a desired position relative to ferrule 16 and feedthrough 18. The desired position may include a position in which respective ones of electrically conductive leads 104 contact internally-facing feedthrough conductive pad 46 and ferrule 16, as shown in FIG. 5.

Once co-fire capacitive filter 82 has been positioned in the desired position relative to ferrule 16 and feedthrough 18, electrically conductive leads 104 may be attached to respective portions of feedthrough 18 and ferrule 16. For example, one of electrically conductive leads 104 may be attached to one of internally-facing feedthrough conductive pads 46 using laser welding, parallel gap welding, thermosonic bonding, diffusion bonding, ultrasonic welding, opposed gap welding, laser brazing, step gap resistance welding, brazed interposer, percussion arc welding, or soldering (conventional or laser). One of electrically conductive leads 104 may be attached to ferrule 16 using a similar process.

In other examples, co-fire capacitive filter 82 may be attached to feedthrough 18 and ferrule 16 using a thick film conductive paste. In some of these examples, attaching co-fire capacitive filter 82 to feedthrough 18 and ferrule 16 (112) may include applying the thick film conductive paste to desired locations of feedthrough 18, ferrule 16, and/or co-fire capacitive filter 82. In some examples, the technique may include the applying thick film conductive paste to one of internally-facing feedthrough conductive pads 46. In other examples, the method may include applying thick film conductive paste to an externally-facing conductive pad on co-fire capacitive filter 82 or capacitive filter conductor 92. In some examples, the technique may include applying thick film conductive paste to an interior wall of ferrule 16. In other examples, the method may include applying thick film conductive paste to capacitive filter perimeter conductive contact 96.

Thick film conductive paste may be applied to the desired locations of feedthrough 18, ferrule 16, and/or co-fire capacitive filter 82 using any one or combination of a variety of techniques, including, for example, screen printing, brushing, using a dispenser, or the like. The thick film conductive paste may initially be in paste form (e.g., a suspension of a powder mixture of Ag, Pd, and, optionally, glass frit in a liquid carrier). In some examples, the amount of liquid carrier may be selected such that the thick film conductive paste is relatively viscous and does not flow readily from the locations at which it is applied (after application). For example, thick film conductive paste may have a viscosity of between about 100 kilocentipoise (kcps; about 1,000 poise) and about 250 (kcps; about 2,500 poise).

Once the thick film conductive paste has been applied to the desired locations of feedthrough 18, ferrule 16, and/or co-fire capacitive filter 82, co-fire capacitive filter 82 may be positioned in a desired orientation relative to ferrule 16 and feedthrough 18. For example, this may include positioning co-fire capacitive filter 82 such that externally-facing filter array side 86 is proximate (near) to internally-facing feedthrough side 38 (e.g., so that the thick film conductive paste is contacting both internally-facing feedthrough conductive pads 46 and capacitive filter conductor 92). This may also include positioning co-fire capacitive filter 82 such that capacitive filter perimeter wall 90 is proximate (near) to the interior wall of ferrule 16 (e.g., so that the thick film conductive paste is contacting both capacitive filter perimeter conductive contact 96 and the interior wall of ferrule 16).

After co-fire capacitive filter 82 has been positioned in the desired orientation relative to ferrule 16 and feedthrough 18, feedthrough assembly 10b may be heated to convert the thick film conductive paste from a paste to a relatively solid (e.g., an Ag—Pd alloy with glass frit) material. For example, feedthrough assembly 10b may be heated at a temperature between about 700° C. and about 850° C. for between about 30 minutes and about 60 minutes, with about 10 minutes maintained at the peak temperature. By heating feedthrough assembly 10b and converting the thick film conductive paste to a relatively solid material, mechanical and electrical connection may be made between the internally-facing feedthrough conductive pad 46 and capacitive filter conductor 92, which may result in mechanical connection between feedthrough 18 and co-fire capacitive filter 82. Similarly, heating feedthrough assembly 10b and converting the thick film conductive paste to a relatively solid material may make mechanical and electrical connection between capacitive filter perimeter conductive contact 96 and the interior wall of ferrule 16, which may result in mechanical connection between ferrule 16 and co-fire capacitive filter 82. In some examples, capacitor array 20 may be attached to feedthrough 18 after co-fire capacitive filter 82 has been attached to feedthrough 18 and ferrule 16. For example, when a thick film conductive paste is used to make electrical connection between co-fire capacitive filter 82 and feedthrough 18 and/or ferrule 16, temperatures that may damage PB 48 may be used to convert the thick film conductive paste to a relatively solid material. Hence, capacitor array 20 may be attached after heating the thick film conductive paste to avoid damage to PB 48 due to heating. In other examples, capacitor array 20 may be attached to feedthrough 18 and/or ferrule before co-fire capacitive filter 82, e.g., when PB 48 is able to withstand without damage the temperatures to which co-fire capacitive filter 82, feedthrough 18 and/or ferrule 16 are heated.

FIGS. 1-3 and 5 illustrate examples of feedthrough assemblies 10a, 10b, in which each conductive pathway through feedthrough 18 is an active conductive pathway (e.g., is electrically connected to a lead conductor. In other examples, at least one conductive pathway through feedthrough 18 may be a ground conductive pathway. FIGS. 7A and 7B are cross-sectional views of an example feedthrough assembly 10c that includes a ground feedthrough conductive pathway 124. The cross-sectional view shown in FIG. 7A was taken along section line A-A in FIG. 2. The cross-sectional view shown in FIG. 7B is taken along section line B-B in FIG. 2. In some examples, feedthrough assembly 10c may be similar to or substantially the same as feedthrough assembly 10a, aside from the differences described herein.

In FIGS. 7A and 7B, feedthrough 18 includes two feedthrough active conductive pathways 122 and one feedthrough ground conductive pathway 124. Feedthrough active conductive pathways 122 may be electrically coupled to respective lead conductors (e.g., see FIG. 20), and thus may conductive electrical signals between externally-facing feedthrough conductive pads 28 and internally-facing feedthrough conductive pads 46. From internally-facing feedthrough conductive pads 46, the electrical signals may be conducted through respective ones of lead wires 52 to respective ones of incoming contact pads 50. The electrical signals may be filtered by respective ones of capacitors 54, conducted to respective ones of outgoing contact pads 56, and conducted to electrical circuitry within the IMD. In this way, feedthrough active conductive pathways 122 provide an electrical pathway through feedthrough assembly 10c.

In accordance with some aspects of the disclosure, capacitors 54 may be electrically coupled to an electrical ground (e.g., a housing of an IMD) through feedthrough ground conductive pathway 124. As shown in FIG. 7B, feedthrough ground conductive pathway 124 may include one of internally-facing feedthrough conductive pads 46, one of feedthrough conductors 44, one of externally-facing feedthrough conductive pads 28, and feedthrough ground conductive via 126. However, in some examples, feedthrough ground conductive pathway 124 may not include one of externally-facing feedthrough conductive pads 28 (e.g., because in some examples, feedthrough ground conductive pathway 124 may not be connected to a lead conductor).

In some examples, feedthrough conductor 44 in feedthrough ground conductive pathway 124 may extend from internally-facing feedthrough side 38 to externally-facing feedthrough side 36. In other examples, feedthrough conductor 44 in feedthrough ground conductive pathway 124 may extend only partially through feedthrough substrate 34 (e.g., from internally-facing feedthrough side 38 partway into feedthrough substrate 34).

Feedthrough ground conductive via 126 may be electrically coupled or connected to feedthrough conductor 44. In some examples, feedthrough ground conductive via 126 may extend between feedthrough conductor 44 and feedthrough perimeter wall 40. As shown in FIG. 7B, feedthrough ground conductive via 126 may be disposed in a plane within feedthrough 18 (e.g., within feedthrough substrate 34). In other examples, feedthrough ground conductive via 126 may be located at alternative locations of feedthrough 18. For example, feedthrough ground conductive via 126 may not be disposed in a single plane within feedthrough substrate 34, but may be disposed in more than one plane within feedthrough substrate 34. In some examples, feedthrough ground conductive via 126 may extend along at least two of the orthogonal x-y-z axes shown in FIG. 7B. In other examples, feedthrough ground conductive via 126 may be disposed on externally-facing side 36 of feedthrough 18 or an internally-facing side 38 of feedthrough 18.

In some examples, feedthrough ground conductive via 126 may be formed of silver, silver-palladium, silver-platinum, or another electrically conductive metal or alloy. As shown in FIG. 7B, feedthrough ground conductive via 126 may be electrically coupled or connected to a feedthrough perimeter conductive contact 128 formed on feedthrough perimeter wall 40. Feedthrough perimeter conductive contact 128 may be formed of an electrically conductive metal or alloy, such as, for example, silver, silver-palladium, or silver-platinum.

In some examples, feedthrough perimeter conductive contact 128 may extend along only a portion of feedthrough perimeter wall 40 (as shown in FIGS. 7A and 7B). For example, feedthrough perimeter conductive contact 128 may extend along feedthrough perimeter wall 40 for a length sufficient to make electrical connection with feedthrough ground conductive via 126 and to facilitate electrical connection with ferrule 16. In other examples, feedthrough perimeter conductive contact 128 may extend substantially along the entire length of feedthrough perimeter wall 40. In some examples, feedthrough 18 may include more than one (e.g., at least two) feedthrough perimeter conductive contact 128.

Feedthrough perimeter conductive contact 128 may be electrically coupled to ferrule 16 via an electrical connection 130. In some examples, electrical connection 130 may include a thick film conductive paste, such as an Ag—Pd paste (with or without glass frit). In other examples, electrical connection 130 may include a solder connection, using, for example, indium-silver (In—Ag) alloys, tin-silver (Sn—Ag), tin-copper (Sn—Cu), tin-silver-copper (Sn—Ag—Cu), tin-lead (Sn—Pb), or gold-tin (Au—Sn). In other examples, electrical connection 130 may include a braze joint.

Ferrule 16 may be electrically coupled or connected to an electrical ground, such as a housing of an IMD (see FIG. 20). Hence, capacitors 54 may be electrically coupled to an electrical ground using feedthrough ground conductive pathway 124 (including feedthrough ground conductive via 126 and perimeter conductive contact 128), and ferrule 16. In this way, EMI signals being filtered by capacitors 54 are grounded.

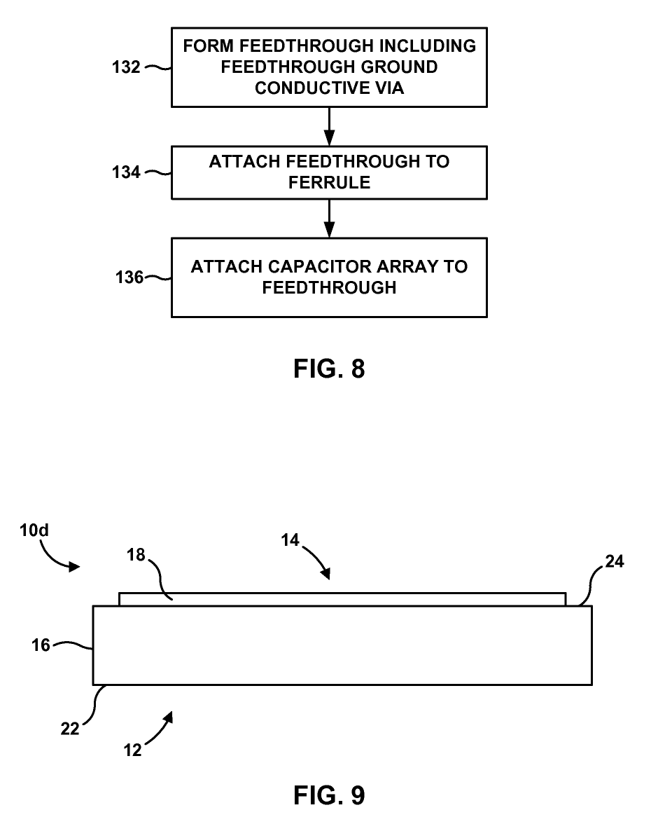

FIG. 8 is a flow diagram that illustrates an example technique for forming a feedthrough assembly in which capacitors 54 are electrically coupled to en electrical ground through feedthrough 18. The technique of FIG. 8 will be described with reference to feedthrough assembly 10c of FIGS. 7A and 7B for clarity. However, the technique may be adapted to form other feedthroughs in accordance with this disclosure.

The technique illustrated in FIG. 8 may optionally include forming a feedthrough 18 that includes a feedthrough conductive via 126 (132). In some examples, feedthrough substrate 34 includes multi-layer ceramic formed from a plurality of generally planar ceramic layers (not shown in FIGS. 7A and 7B). In some examples, the ceramic may include a high-temperature cofired ceramic (HTCC) material, e.g., a ceramic that is sintered at a temperature of at least about 1300° C., for example a material that is sintered at a temperature of at least about 1600° C. In some implementations, HTCC uses an electrical insulator that includes alumina and may include oxides of Si (silica), Ca (calcia), Mg (magnesia), Zr (zirconia), or the like. In some examples, feedthrough substrate 20 may comprise at least about 70% alumina, for example at least about 90% alumina having a sintering temperature of between about 1550° C. and about 1600° C.

Feedthrough 18 also includes feedthrough conductors 44 and feedthrough ground conductive via 126. Feedthrough conductors 44 and feedthrough ground conductive via 126 may be formed of a metal or alloy. For example, feedthrough conductors 44 and feedthrough ground conductive via 76 may be formed of copper (Cu), silver (Ag), gold (Au), platinum (Pt), palladium (Pd), niobium (Nb), iridium (Ir), titanium (Ti), tungsten (W), molybdenum (Mb), zirconium (Zr), osmium (Os), tantalum (Ta), vanadium (V), rhodium (Rh), rhenium (Re), and ruthenium (Ru), platinum-gold alloys, platinum-iridium alloys, platinum-palladium alloys, gold-palladium alloys, titanium alloys, such as Ti-6Al-4V, Ti-45Nb, Ti-15Mo or titanium-vanadium, or tungsten-molybdenum alloys.

In some examples, feedthrough conductors 44 and/or feedthrough ground conductive via 126 may be formed of a conductive paste that is used to fill via holes to form feedthrough conductors 44 and/or be deposited on feedthrough substrate 34 to form feedthrough ground conductive via 126. The conductive paste may include, for example, a metallic paste that is applied to the via holes, for example a platinum-containing paste, a tungsten-containing paste, Nb-containing paste, Ta-containing paste, Au-containing paste, or a molymanganese-containing paste. Such materials may be biocompatible and biostable materials. In one example, the metallic paste primarily comprises a metallic powder, such as platinum or palladium powder, and an additive to promote bonding with the material of feedthrough substrate 34. The additive may also provide for thermal expansion compatibility between the conductive paste and the HTCC material of feedthrough substrate 34. In one example, the additive comprises alumina, so that the metallic paste may comprise, for example, a majority of metallic powder, such as platinum powder, and a minority of alumina powder or particles mixed therein.

In examples in which feedthrough substrate 34 is formed from multiple ceramic layers, each ceramic layer may be shaped in a green state to have a layer thickness and a plurality of via holes extending there through between an internally facing layer surface and an externally facing layer surface. The ceramic layers then may be coupled together, such as by laminating the layers together, and may be cofired together so that the layers form a substantially monolithic feedthrough substrate 34. In some examples, the via holes of each ceramic layer may be substantially aligned to form generally cylindrical passages that are filled with an electrically conductive material to form feedthrough conductors 44. In examples in which feedthrough ground conductive via 126 is formed in a plane within feedthrough substrate 34, the material from which feedthrough ground conductive via 76 is formed may be deposited on a ceramic layer prior to cofiring the ceramic layers together. In examples in which feedthrough ground conductive via 126 is formed on externally-facing feedthrough side 36 or internally-facing feedthrough side 38, the material from which feedthrough ground conductive via 126 is formed may be deposited on a ceramic layer before or after firing the ceramic layers together.

The technique of FIG. 8 also includes attaching feedthrough 18 to ferrule 16 (134). Feedthrough 18 may be connected to ferrule 16 using any technique that forms hermetic seal 26 between feedthrough 18 and ferrule 16. For example, ferrule 16 and feedthrough 18 may be connected using brazing, diffusion bonding, laser sintering of glass, or the like. Hermetic seal 26 may be formed using a biocompatible, biostable material. Examples of materials that may be used to form a hermetic seal 26 include gold, a nickel-gold alloy, platinum, platinum-iridium, or a biocompatible glass.

Additionally, attaching feedthrough 18 to ferrule 16 (134) may include electrically attaching feedthrough ground conductive via 126 to ferrule 16. As described above, in some examples, this may include using a weld, braze, solder, or thick film conductive paste to form electrical connection 130 between feedthrough ground conductive via 126 to ferrule 16 (or between feedthrough perimeter conductive contact 128 and ferrule 16).

The technique illustrated in FIG. 8 also may include attaching capacitor array 20 to feedthrough 18 (136). In some examples, attaching capacitor array 20 to feedthrough 18 may include attaching lead wires 52 to feedthrough 18 (74), and attaching lead wires 52 to incoming contact pads 50 of capacitor array 20 (76), as described above with respect to FIG. 4. As described above, lead wires 52 may be attached to internally-facing feedthrough conductive pads 46 (or feedthrough conductors 44) by soldering, welding, or the like. Similarly, lead wires 52 may be attached to incoming contact pads 50 using soldering, welding, or the like.

The technique of FIG. 8 also may include attaching ground lead wire 66 to feedthrough ground conductive pathway 124 and outgoing ground contact pad 64. Ground lead wire 66 may be attached to feedthrough ground conductive pathway 124 and/or outgoing ground contact pad 64 by soldering, welding, or the like.

In some examples, a feedthrough assembly may include individual capacitors welded directly to feedthrough 18 (or attached to leads welded to feedthrough 18) instead of or in addition to including a capacitor array 20 (which includes capacitors 54 mounted on a PB 48). In some examples, attaching individual capacitors directly to feedthrough 18 may reduce a length of signal paths for the electrical signals transmitted through the feedthrough assembly (compared to using a capacitor array 20). Additionally, in some examples, by attaching individual capacitors directly to feedthrough 18, the capacitors may occupy a smaller volume of space than capacitor array 20.

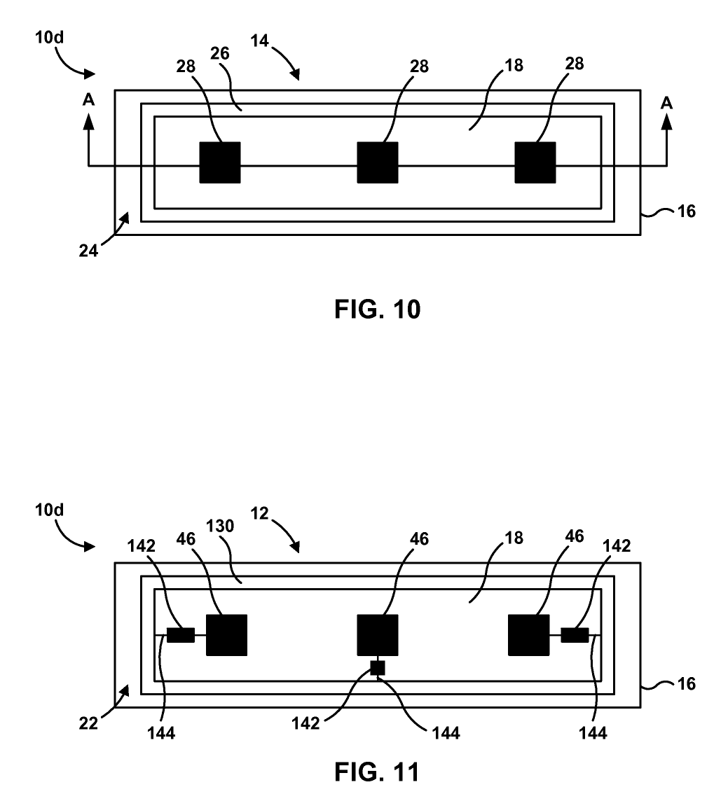

FIGS. 9-12 illustrate an example feedthrough assembly 10d that includes chip capacitors 142. FIG. 9 is a side view of feedthrough assembly 10d and shows feedthrough 18 and ferrule 16. FIG. 10 is a top view of feedthrough assembly 10d, which shows externally-facing side 14 of feedthrough assembly 10d. FIG. 11 is a bottom view of feedthrough assembly 10d, which shows internally-facing side 12 of feedthrough assembly 10d.

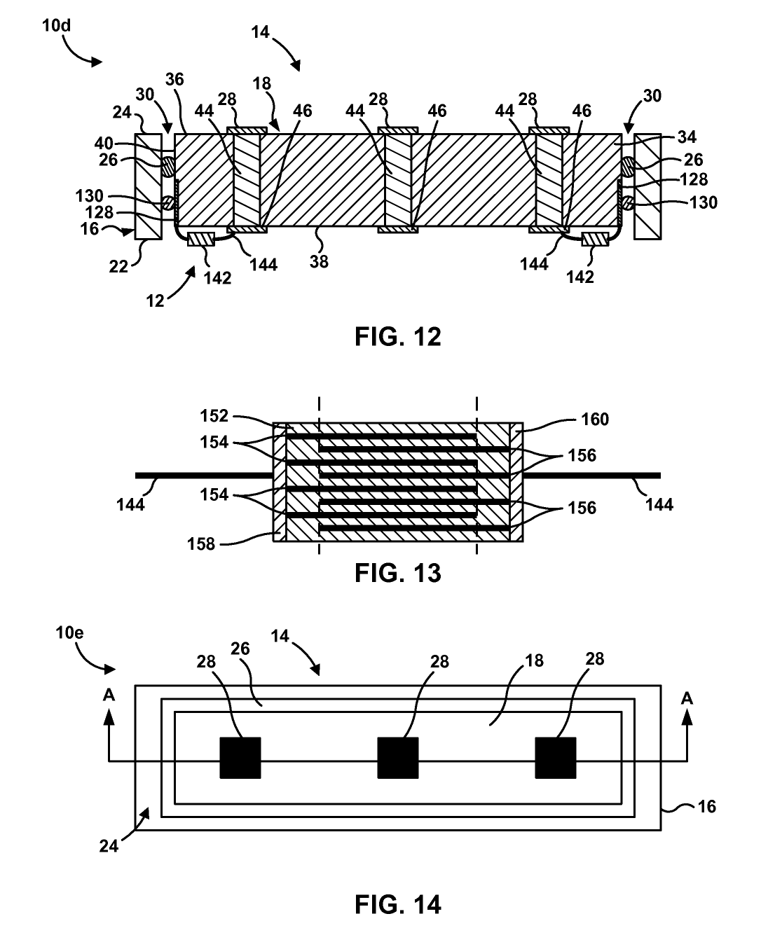

Ferrule 16 may be similar to or substantially the same as ferrule 16 described above with respect to FIGS. 1-3, 5, 7A, and 7B. Feedthrough 18 may be similar to feedthroughs 18 described above with respect to FIGS. 1-3 and 5. However, in contrast to the examples illustrated in FIGS. 1-3 and 5, feedthrough 18 in FIGS. 9-12 includes a feedthrough perimeter conductive contact 128 (shown in FIG. 12).

As described above with respect to FIGS. 7A and 7B, feedthrough perimeter conductive contact 128 may be formed of an electrically conductive metal or alloy, such as, for example, silver, silver-palladium, or silver-platinum. In some examples, feedthrough perimeter conductive contact 128 may extend along only a portion of feedthrough perimeter wall 40 (as shown in FIGS. 7A and 7B). For example, feedthrough perimeter conductive contact 128 may extend along feedthrough perimeter wall 40 for a length sufficient to make electrical connection with one or more of leads 144 and to facilitate electrical connection with ferrule 16. In other examples, feedthrough perimeter conductive contact 128 may extend substantially along the entire length of feedthrough perimeter wall 40. In some examples, feedthrough 18 may include more than one (e.g., at least two) feedthrough perimeter conductive contact 128.

Feedthrough perimeter conductive contact 128 may be electrically coupled to ferrule 16 via an electrical connection 130. In some examples, electrical connection 130 may include a thick film conductive paste, such as an Ag—Pd paste (with or without glass frit). In other examples, electrical connection 130 may include a solder connection, using, for example, indium-silver (In—Ag) alloys, tin-silver (Sn—Ag), tin-copper (Sn—Cu), tin-silver-copper (Sn—Ag—Cu), tin-lead (Sn—Pb), or gold-tin (Au—Sn). In other examples, electrical connection 130 may include a braze joint

Instead of including a capacitor array 20 and/or a co-fire capacitive filter 82, feedthrough assembly 10d includes chip capacitors 142. Respective ones of chip capacitors 142 are attached directly to respective ones of internally-facing feedthrough conductive pads 46 and feedthrough perimeter conductive contact 128 using leads 144.

In some examples, chip capacitors 142 may include, for example, multilayer ceramic chip capacitors. FIG. 13 is a cross-sectional diagram that illustrates an example chip capacitor 142 that is a multilayer ceramic chip capacitor. As shown in FIG. 13, chip capacitor 142 may include alternating layers of a ceramic dielectric material 152 and metal electrodes 154, 156. Ceramic dielectric material 152 may include any electrically insulative ceramic, such as barium titanate (BaTiO₃) or alumina (Al₂O₃). In some examples, ceramic dielectric material 152 may include barium titanate (BaTiO₃) or alumina and at least one of silica (SiO₂), calcia (CaO), magnesia (MgO), zirconia (ZrO₂), and the like.

Metal electrodes 154, 156 may include an electrically conductive metal or alloy, such as copper (Cu), silver (Ag), gold (Au), nickel (Ni), platinum (Pt), palladium (Pd), niobium (Nb), iridium (Ir), titanium (Ti), tungsten (W), molybdenum (Mb), zirconium (Zr), osmium (Os), tantalum (Ta), vanadium (V), rhodium (Rh), rhenium (Re), and ruthenium (Ru), and alloys, mixtures, and combinations thereof. In some examples, metal electrodes 154, 156 may include a palladium-silver alloy or nickel.

Metal electrodes 154 electrically connect to a first termination 158. Termination 158 may be formed of an electrically conductive material, such as a metal or alloy. In some examples, termination 158 includes a single layer of conductive material. In other examples, termination 158 may include at least two layers. For example, a first layer may include copper (Cu) and/or nickel (Ni), a second layer may include nickel (Ni), and a third layer may include tin (Sn). Termination 158 may be electrically connected to one of leads 144, e.g., by welding or soldering.

Metal electrodes 156 electrically connect to a second termination 160. Second termination 160 may be similar to first termination 158, and may include an electrically conductive material. Second termination 160 may include a single layer or at least two layers, as described with respect to first termination 158. Second termination 160 is electrically connected to one of leads 144, e.g., by welding or soldering.

Leads 144 may include any electrically conductive material, and may in some examples include a metal or alloy, such as copper (Cu), silver (Ag), gold (Au), nickel (Ni), platinum (Pt), palladium (Pd), niobium (Nb), iridium (Ir), titanium (Ti), tungsten (W), molybdenum (Mb), zirconium (Zr), osmium (Os), tantalum (Ta), vanadium (V), rhodium (Rh), rhenium (Re), and ruthenium (Ru), and alloys, mixtures, and combinations thereof.

As shown in FIG. 12, respective ones of leads 144 may be directly attached to respective ones of internally-facing feedthrough conductive pads 46 and to feedthrough perimeter conductive contact 128. For example, respective ones of leads 144 may be directly attached to respective ones of internally-facing feedthrough conductive pads 46 and to feedthrough perimeter conductive contact 128 using laser welding, parallel gap welding, thermosonic bonding, diffusion bonding, ultrasonic welding, opposed gap welding, laser brazing, step gap resistance welding, brazed interposer, percussion arc welding, or soldering (conventional or laser). In this way, electrical signals conducted by each of the feedthrough conductive pathways are filtered using respective chip capacitors 142. Hence, chip capacitors 142 may reduce or substantially eliminate electrical interference due to signals inadvertently sensed and conducted by the lead conductors, and may facilitate or protect operation of the electronics in the IMD.

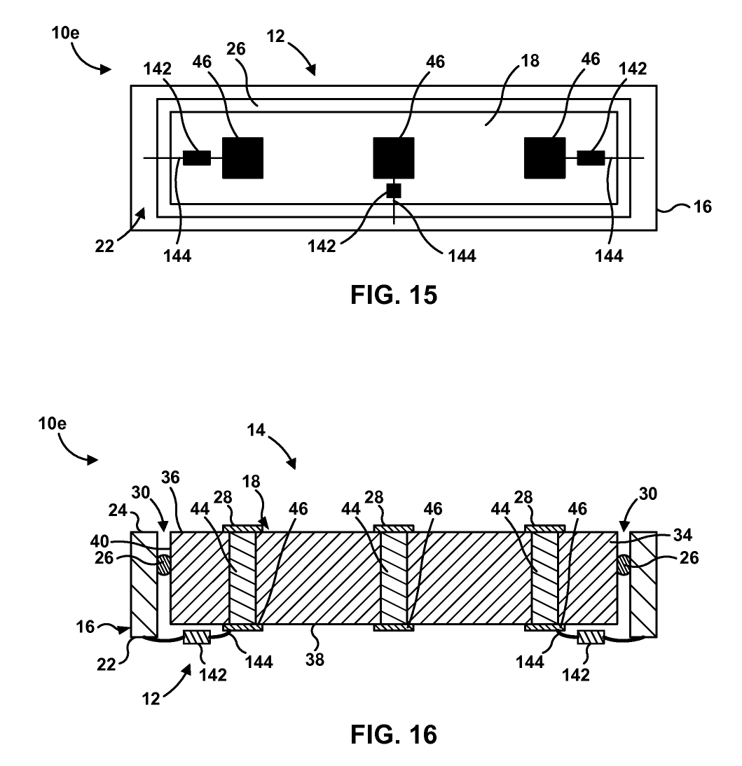

In some examples, as shown in FIGS. 14-16, a feedthrough assembly 10e may include chip capacitors 142 connected to leads 144. Feedthrough assembly 10e may be similar to or substantially the same as feedthrough assembly 10d aside from the differences described herein. For example, instead of respective ones of leads 144 being electrically connected to feedthrough perimeter conductive contact 128, as shown in FIG. 12, respective ones of leads 144 may be electrically connected to ferrule 16. Respective ones of leads 144 may be electrically connected to ferrule 16 laser welding, parallel gap welding, thermosonic bonding, diffusion bonding, ultrasonic welding, opposed gap welding, laser brazing, step gap resistance welding, brazed interposer, percussion arc welding, or soldering (conventional or laser). The configuration shown in FIGS. 14-16 may simplify construction of feedthrough 18 (compared to feedthrough 18 in feedthrough assembly 10d), as feedthrough 18 in feedthrough assembly 10e may not include perimeter conductive contact 128.

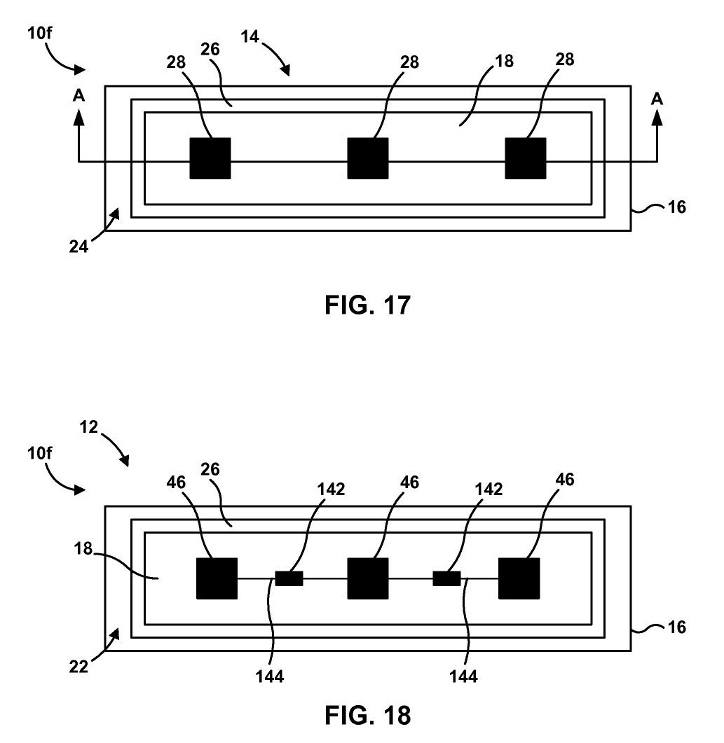

FIGS. 17-19 illustrate another example feedthrough assembly 10f. Feedthrough assembly 10f may be similar to or substantially the same as feedthrough assembly 10d aside from the differences described herein. In contrast to feedthrough assembly 10d, feedthrough assembly 10e includes a feedthrough 18 that includes two feedthrough active conductive pathways 122 and one feedthrough ground conductive pathway 124. In some examples, feedthrough 18 in feedthrough assembly 10f may be similar to feedthrough 18 in feedthrough assembly 10c (FIGS. 7A and 7B), and feedthrough ground conductive pathway 124 may be electrically connected to a feedthrough ground conductive via 126 and, optionally, a feedthrough perimeter conductive contact 128.

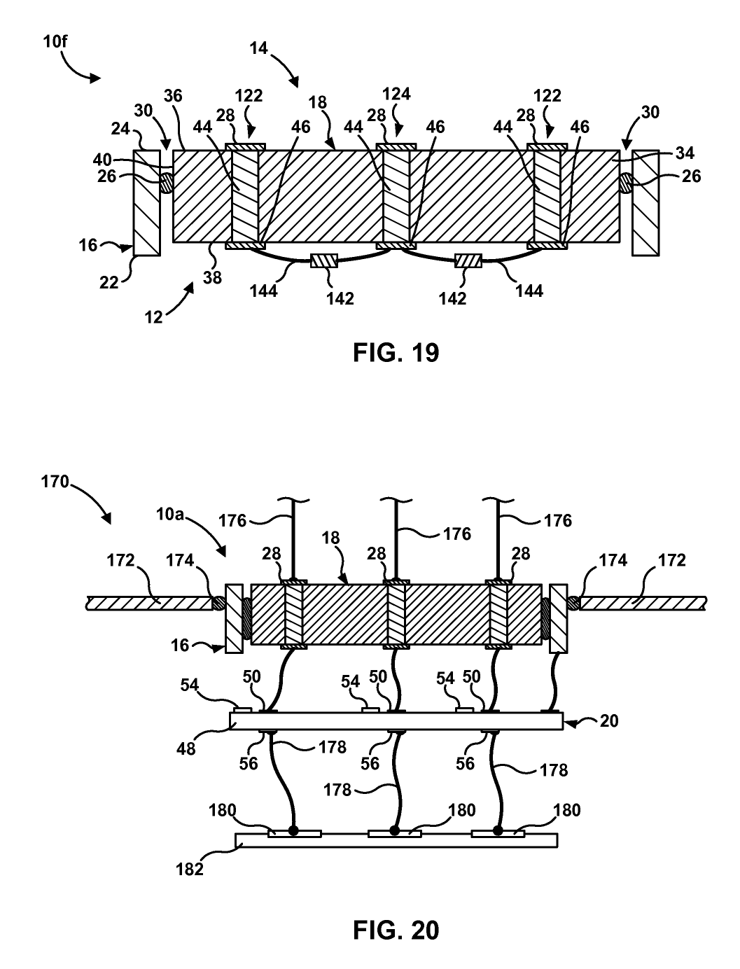

As shown in FIGS. 18 and 19, respective ones of leads 144 may be electrically connected to feedthrough active conductive pathways 122 and feedthrough ground conductive pathway 124 through respective ones internally-facing feedthrough conductive pads 46. The connection of respective leads 144 to respective ones feedthrough active conductive pathways 122 and feedthrough ground conductive pathway 124 may result in respective chip capacitors 142 being electrically connected between a respective one of feedthrough active conductive pathways 122 and feedthrough ground conductive pathway 124. In this way, electrical signals conducted by each of the feedthrough active conductive pathways 122 are filtered using respective chip capacitors 142.

Although chip capacitors 142 are electrically connected to feedthrough 18 and/or ferrule 16 using leads 144 in feedthrough assemblies 10d, 10e, and 10f, this may not be the case in all examples. For example, in some implementations, chip capacitors 142 may be directly electrically connected to respective ones of internally-facing feedthrough conductive pads 46, respective ones of feedthrough perimeter conductive contact 128, and/or ferrule 16 (e.g., without using leads 144). For example, first termination 158 may be welded, soldered, or brazed directly to one of internally-facing feedthrough conductive pads 46 and second termination 160 may be welded, soldered, or brazed directly to another of internally-facing feedthrough conductive pads 46. As another example, first termination 158 may be welded, soldered, or brazed directly to one of internally-facing feedthrough conductive pads 46 and second termination 160 may be welded, soldered, or brazed directly to feedthrough perimeter conductive contact 128. As a further example, first termination 158 may be welded, soldered, or brazed directly to one of internally-facing feedthrough conductive pads 46 and second termination 160 may be welded, soldered, or brazed directly to ferrule 16.

In any of the feedthrough assemblies 10d, 10e, 10f, electrical connectors, such as wires, may be electrically connected to internally-facing feedthrough conductive pads 46 (e.g., in the feedthrough active conductive pathways 122) to electrically connect feedthrough 18 to circuitry within the IMD in which feedthrough assemblies 10d, 10e, 10f are used.

Any of the feedthrough assemblies 10a, 10b, 10c, 10d, 10e, 10f (collectively, “feedthrough assembly 10”) illustrated and described above may be utilized as a feedthrough assembly for an IMD. FIG. 20 is a conceptual diagram that illustrates an example feedthrough assembly attached to a housing of an IMD and electrically coupled to a plurality of leads and a plurality of electrical connections to circuitry. Although feedthrough assembly 10a is depicted in FIG. 20, any of the other feedthrough assemblies described herein may be utilized in an IMD in a similar manner.

IMD 170 includes a housing 172 and defines an opening in which feedthrough assembly 10a is disposed. Feedthrough assembly 10a is mechanically attached to a housing 172 of IMD 170 by a hermetic seal 174. For example, hermetic seal 174 may be formed between an exterior wall of ferrule 16 and housing 172. Hermetic seal 174 may prevent bodily fluids of the patient from passing into the interior of IMD housing between ferrule 16 and housing 172, which could lead to damage to the internal electronics of the IMD 170. In one example, hermetic seal 174 comprises a braze joint between ferrule 16 and housing 172 (e.g., formed using laser brazing). In other examples, hermetic seal 174 may be formed using diffusion bonding. Examples of materials that may be used to form a hermetic seal 174 include any biocompatible, biostable material capable for forming a hermetic seal 174, such as, gold, a nickel-gold alloy, platinum, and platinum-iridium. Laser sintering of glass may also be used to bond ferrule 16 and housing 172.

In other examples, hermetic seal 174 may include a weld formed between housing 162 and ferrule 16. The weld may be formed of a material that is compatible with the material of housing 172 and the material of ferrule 16. As described above, in some examples, ferrule 16 may include titanium or a titanium alloy, and housing 172 also may include a titanium or titanium alloy. In some examples, the weld is formed using a laser welding process, e.g., to form a Ti—Ti weld.

In some examples, hermetic seal 174 may provide an electrical connection between housing 172 and ferrule 16 and may form a portion of the electrically conductive path between capacitors 54 and housing 172. In some of these examples, housing 172 may act as an electrical ground for the signals filtered by capacitors 54.

In some examples, IMD 170 may be device that is configured to deliver a therapy and/or monitor a physiologic condition of a patient. For example, IMD 170 may be a cardiac pacemaker, an implantable cardioverter/defibrillator, or an implantable neurostimulator, and may deliver therapy to or monitor physiologic signals from a patient's heart, muscle, nerve, brain, stomach, or another organ.

IMD 170 encloses circuitry, such as therapy delivery circuitry or sensing circuitry. Therapy delivery circuitry and/or sensing circuitry are represented in FIG. 20 as a printed board (PB) 182. Although not shown in FIG. 20, PB 182 may include electrical components, such as resistors, capacitor, inductors, batteries, integrated circuits, hybrid circuits, analog circuits, or the like mounted to or incorporated into PB 182. PB 182 also includes a plurality of contact pads 180, to which wires 178 are electrically connected.

Wires 178 electrically connect circuitry in or on PB 182 to outgoing contact pads 56. Respective wires 178 may be electrically connected to respective contact pads 180 and respective outgoing contact pads 56. Electrical connection between wires 178 and contact pads 180 and respective outgoing contact pads 56 may be made by, for example, welding or soldering.

IMD 170 is also electrically connected to a plurality of lead conductors 176 via feedthrough assembly 10a. For example, respective ones of lead conductors 176 may be electrically connected to respective ones of externally-facing feedthrough conductive pads 28. Lead conductors 176 may be carried by at least one lead body, which may also carry electrodes, to which lead conductors 176 are electrically connected. Lead conductors 176 provide an electrical path through which IMD 170 may deliver electrical stimulation of a target tissue and/or sense physiologic signals from a target tissue.

Various examples have been described. These and other examples are within the scope of the following claims.

1. A feedthrough assembly comprising:
a ferrule defining a ferrule opening;
a feedthrough at least partially disposed within the ferrule opening and attached to the ferrule, wherein the feedthrough comprises a plurality of feedthrough conductive pathways extending between an externally-facing side of the feedthrough and an internally-facing side of the feedthrough; and
a plurality of chip capacitors, wherein respective ones of the plurality of chip capacitors are electrically connected to respective ones of the plurality of feedthrough conductive pathways and electrically connected to the ferrule.

2. The feedthrough assembly of claim 1, wherein respective ones of the plurality of chip capacitors are directly electrically connected to respective ones of the plurality of feedthrough conductive pathways using respective ones of a plurality of leads.

3. The feedthrough assembly of claim 1, wherein at least one of the plurality of chip capacitors is directly electrically connected to one of the plurality of feedthrough conductive pathways by welding, soldering or brazing a termination of the at least one of the plurality of chip capacitors directly to the one of the plurality of feedthrough conductive pathways.

4. The feedthrough assembly of claim 1, wherein the feedthrough further comprises a feedthrough perimeter conductive contact electrically connected to the ferrule, and wherein respective ones of the plurality of chip capacitors are electrically connected to the feedthrough perimeter conductive contact.

5. The feedthrough assembly of claim 4, wherein at least one of the plurality of chip capacitors is directly electrically connected to the feedthrough perimeter conductive contact using a respective one of a plurality of leads.

6. The feedthrough assembly of claim 4, wherein at least one of the plurality of chip capacitors is directly electrically connected to the feedthrough perimeter conductive contact by welding, soldering or brazing a termination of the at least one of the plurality of chip capacitors directly to the feedthrough perimeter conductive contact.

7. The feedthrough assembly of claim 1, wherein respective ones of the plurality of chip capacitors are directly electrically connected to the ferrule using a respective one of a plurality of leads.

8. The feedthrough assembly of claim 1, wherein at least one of the plurality of chip capacitors is directly electrically connected to the ferrule by welding, soldering or brazing a termination of the at least one of the plurality of chip capacitors directly to the ferrule.

9. The feedthrough assembly of claim 1, wherein the plurality of feedthrough conductive pathways comprises a feedthrough active conductive pathway and a feedthrough ground conductive pathway, wherein the feedthrough ground conductive pathway is electrically connected to the ferrule, and wherein at least one of the plurality of chip capacitors is electrically connected between the feedthrough active conductive pathway and the feedthrough ground conductive pathway.

10. The feedthrough assembly of claim 9, wherein the at least one of the plurality of chip capacitors is directly electrically connected to the feedthrough active conductive pathway using a respective one of a plurality of leads and is directly electrically connected to the feedthrough ground conductive pathway using another respective one of the plurality of leads.

11. The feedthrough assembly of claim 9, wherein the at least one of the plurality of chip capacitors is directly electrically connected to the feedthrough active conductive pathway by welding, soldering, or brazing a first termination of the at least one of the plurality of chip capacitors directly to the feedthrough active conductive pathway and is directly electrically connected to the feedthrough ground conductive pathway by welding, soldering, or brazing a second termination of the at least one of the plurality of chip capacitors directly to the feedthrough ground conductive pathway.

12. An implantable medical device comprising:
a housing defining an opening; and
a feedthrough assembly disposed in the opening and attached to the housing, wherein the feedthrough assembly comprises:
a ferrule defining a ferrule opening;
a feedthrough at least partially disposed within the ferrule opening and attached to the ferrule, wherein the feedthrough comprises a plurality of feedthrough conductive pathways extending between an externally-facing side of the feedthrough and an internally-facing side of the feedthrough; and
a plurality of chip capacitors, wherein respective ones of the plurality of chip capacitors are electrically connected to respective ones of the plurality of feedthrough conductive pathways and electrically connected to the ferrule.

13. The implantable medical device of claim 12, wherein respective ones of the plurality of chip capacitors are directly electrically connected to respective ones of the plurality of feedthrough conductive pathways using respective ones of a plurality of leads.

14. The implantable medical device of claim 12, wherein at least one of the plurality of chip capacitors is directly electrically connected to one of the plurality of feedthrough conductive pathways by welding, soldering or brazing a termination of the at least one of the plurality of chip capacitors directly to the one of the plurality of feedthrough conductive pathways.

15. The implantable medical device of claim 12, wherein the feedthrough further comprises a feedthrough perimeter conductive electrically connected to the ferrule, and wherein respective ones of the plurality of chip capacitors are electrically connected to the feedthrough perimeter conductive contact.

16. The implantable medical device of claim 15, wherein at least one of the plurality of chip capacitors is directly electrically connected to the feedthrough perimeter conductive contact using a respective one of a plurality of leads.

17. The implantable medical device of claim 15, wherein at least one of the plurality of chip capacitors is directly electrically connected to the feedthrough perimeter conductive contact by welding, soldering or brazing a termination of the at least one of the plurality of chip capacitors directly to the feedthrough perimeter conductive contact.

18. The implantable medical device of claim 12, wherein respective ones of the plurality of chip capacitors are directly electrically connected to the ferrule using a respective one of a plurality of leads.

19. The implantable medical device of claim 12, wherein at least one of the plurality of chip capacitors is directly electrically connected to the ferrule by welding, soldering or brazing a termination of the at least one of the plurality of chip capacitors directly to the ferrule.

20. The implantable medical device of claim 12, wherein the plurality of feedthrough conductive pathways comprises a feedthrough active conductive pathway and a feedthrough ground conductive pathway, wherein the feedthrough ground conductive pathway is electrically connected to the ferrule, and wherein at least one of the plurality of chip capacitors is electrically connected between the feedthrough active conductive pathway and the feedthrough ground conductive pathway.

21. The implantable medical device of claim 20, wherein the at least one of the plurality of chip capacitors is directly electrically connected to the feedthrough active conductive pathway using a respective one of the plurality of leads and is directly electrically connected to the feedthrough ground conductive pathway using a respective one of the plurality of leads.

22. The implantable medical device of claim 20, wherein the at least one of the plurality of chip capacitors is directly electrically connected to the feedthrough active conductive pathway by welding, soldering, or brazing a first termination of the at least one of the plurality of chip capacitors directly to the feedthrough active conductive pathway and is directly electrically connected to the feedthrough ground conductive pathway by welding, soldering, or brazing a second termination of the at least one of the plurality of chip capacitors directly to the feedthrough ground conductive pathway.

23. The implantable medical device of claim 12, further comprising a hermetic seal between the feedthrough and the ferrule.

24. The implantable medical device of claim 12, wherein the ferrule is electrically connected to the housing.

25. The implantable medical device of claim 12, further comprising a hermetic seal between the ferrule and the housing.

26. A method comprising:
attaching a perimeter wall of a feedthrough to an interior wall of a ferrule to form a hermetic seal between the feedthrough and the ferrule, wherein the feedthrough comprises a plurality of feedthrough conductive pathways extending between an externally-facing side of the feedthrough and an internally-facing side of the feedthrough; and
electrically connecting a respective one of a plurality of chip capacitors between a respective one of the plurality of feedthrough conductive pathways and the ferrule.

27. The method of claim 26, wherein electrically connecting the respective one of the plurality of chip capacitors between the respective one of the plurality of feedthrough conductive pathways and the ferrule comprises directly electrically connecting the respective one of the plurality of chip capacitors between the respective one of the plurality of feedthrough conductive pathways using respective ones of a plurality of leads.

28. The method of claim 26, wherein electrically connecting the respective one of the plurality of chip capacitors between the respective one of the plurality of feedthrough conductive pathways and the ferrule comprises directly electrically connecting at least one of the plurality of chip capacitors to one of the plurality of feedthrough conductive pathways by welding, soldering or brazing a termination of the at least one of the plurality of chip capacitors directly to the one of the plurality of feedthrough conductive pathways.

29. The method of claim 26, wherein the feedthrough further comprises a feedthrough perimeter conductive contact electrically connected to the ferrule, and wherein electrically connecting the respective one of the plurality of chip capacitors between the respective one of the plurality of feedthrough conductive pathways and the ferrule comprises electrically connecting respective ones of the plurality of chip capacitors to the feedthrough perimeter conductive contact.

30. The method of claim 29, wherein electrically connecting respective ones of the plurality of chip capacitors to the feedthrough perimeter conductive contact comprises directly electrically connecting at least one of the plurality of chip capacitors to the feedthrough perimeter conductive contact using a respective one of a plurality of leads.

31. The method of claim 29, wherein electrically connecting respective ones of the plurality of chip capacitors to the feedthrough perimeter conductive contact comprises directly electrically connecting at least one of the plurality of chip capacitors to the feedthrough perimeter conductive contact by welding, soldering or brazing a termination of the at least one of the plurality of chip capacitors directly to the feedthrough perimeter conductive contact.

32. The method of claim 26, wherein electrically connecting the respective one of the plurality of chip capacitors between the respective one of the plurality of feedthrough conductive pathways and the ferrule comprises directly electrically connecting respective ones of the plurality of chip capacitors to the ferrule using a respective one of a plurality of leads.

33. The method of claim 26, wherein electrically connecting the respective one of the plurality of chip capacitors between the respective one of the plurality of feedthrough conductive pathways and the ferrule comprises directly electrically connecting at least one of the plurality of chip capacitors to the ferrule by welding, soldering or brazing a termination of the at least one of the plurality of chip capacitors directly to the ferrule.

34. The method of claim 26, wherein the plurality of feedthrough conductive pathways comprises a feedthrough active conductive pathway and a feedthrough ground conductive pathway, wherein the feedthrough ground conductive pathway is electrically connected to the ferrule, and wherein electrically connecting the respective one of the plurality of chip capacitors between the respective one of the plurality of feedthrough conductive pathways and the ferrule comprises electrically connecting at least one of the plurality of chip capacitors between the feedthrough active conductive pathway and the feedthrough ground conductive pathway.

35. The method of claim 34, wherein electrically connecting at least one of the plurality of chip capacitors between the feedthrough active conductive pathway and the feedthrough ground conductive pathway comprises:
directly electrically connecting the at least one of the plurality of chip capacitors to the feedthrough active conductive pathway using a respective one of a plurality of leads, and
directly electrically connecting the at least one of the plurality of chip capacitors to the feedthrough ground conductive pathway using a respective one of the plurality of leads.

36. The method of claim 34, wherein electrically connecting at least one of the plurality of chip capacitors between the feedthrough active conductive pathway and the feedthrough ground conductive pathway comprises:
directly electrically connecting the at least one of the plurality of chip capacitors to the feedthrough active conductive pathway by welding, soldering, or brazing a first termination of the at least one of the plurality of chip capacitors directly to the feedthrough active conductive pathway,
and directly electrically connecting the at least one of the plurality of chip capacitors to the feedthrough ground conductive pathway by welding, soldering, or brazing a second termination of the at least one of the plurality of chip capacitors directly to the feedthrough ground conductive pathway.

37. The method of claim 26, further comprising electrically connecting the ferrule to a housing of an implantable medical device.

38. The method of claim 26, further comprising forming a hermetic seal between the ferrule and a housing of an implantable medical device.
